(19) United States
(12) Patent Application Publication
(10) Pub. No.: US 2024/0316049 A1
(43) Pub. Date: Sep. 26, 2024

(54) STABILIZATION OF PHENOBARBITAL SODIUM FOR INJECTION

(71) Applicant: SUN PHARMA ADVANCED RESEARCH COMPANY LIMITED, Andhen (East), Mumbai (IN)

(72) Inventors: Malay SHAH, Vadodara (IN); Bhushan BOROLE, Jalgaon (IN); Ravi PATEL, Anand (IN); Ajay Jaysingh KHOPADE, Vadodara (IN)

(21) Appl. No.: 18/122,008

(22) Filed: Mar. 15, 2023

Related U.S. Application Data
(63) Continuation-in-part of application No. 17/715,491, filed on Apr. 7, 2022, now Pat. No. 11,857,683.
(60) Provisional application No. 63/319,918, filed on Mar. 15, 2022.

(30) Foreign Application Priority Data
Jul. 7, 2021 (IN) 202121030559

(51) Int. Cl. A61K 31/515; A61K 9/00; A61K 9/19; A61K 47/08

(52) U.S. Cl. CPC A61K 31/515; A61K 9/0019; A61K 9/19; A61K 47/08

(57) ABSTRACT

The present invention relates to a lyophilized pharmaceutical composition of hydrolytically unstable pharmaceutical compounds, such as phenobarbital or salts thereof. The present invention also relates to an aqueous solution for injection of phenobarbital or salts thereof that is reconstituted from the lyophilized pharmaceutical composition. The pharmaceutical compositions of the present disclosure have an ethanol content in the range from about 12000 ppm to about 25000 ppm. The composition of the present disclosure, in certain embodiments, is stable following 36 months of storage, wherein the total impurities do not exceed 0.2%. The pharmaceutical compositions of the present disclosure may be used for the treatment of neonatal seizures. The present invention further relates to methods of preparing a lyophilized composition of phenobarbital sodium and to lyophilized pharmaceutical compositions of phenobarbital sodium prepared using the same.

### CROSS REFERENCE TO RELATED APPLICATIONS

The present application claims priority to U.S. patent application Ser. No. 17/715,491, filed on Apr. 7, 2022, which claims priority to U.S. Provisional Patent Application No. 63/319,918 filed on Mar. 15, 2022, the contents of which are incorporated herein in their entireties.

### FIELD OF THE INVENTION

The present invention relates to a lyophilized pharmaceutical composition of hydrolytically unstable pharmaceutical compounds, such as phenobarbital or salts thereof. The present invention also relates to an aqueous solution for injection of phenobarbital or salts thereof that is reconstituted from the lyophilized pharmaceutical composition. The pharmaceutical compositions of the present disclosure have an ethanol content in the range from about 5000 ppm to about 70000 ppm. The compositions of the present disclosure, in certain embodiments, are stable following two years of storage, wherein the total impurities do not exceed 0.5%. The pharmaceutical compositions of the present disclosure may be used for the treatment of neonatal seizures.

### BACKGROUND OF THE INVENTION

Phenobarbital is an anti-epileptic drug which has been used for many years in the treatment of neonatal seizures. One of the problems associated with a phenobarbital composition, however, is its instability due to hydrolysis, which causes it to possess a higher level of impurities. While the compound is freely soluble in water, with solubility reported to be as high as 333 mg/ml to 1 g/ml, the presence of hydroxyl ions from the water in the composition results in a hydrolysis pathway that can destroy the phenobarbital ring complex. This destruction results in the possible formation of impurities including harmful degradants or precipitates.

One known method of improving the stability of phenobarbital sodium involves the use of a lower pH and the use of a mixture of water and generic solvents such as alcohol, propylene glycol, glycerin, benzyl alcohol, or polyethylene glycol. Currently, marketed compositions of phenobarbital sodium injections contain benzyl alcohol, alcohol, and propylene glycol, which pose a higher toxicity risk to neonates and therefore present a potential safety risk when used in the treatment of neonatal seizures. Nevertheless, neonatologists have no choice but to give benzyl alcohol-, alcohol-, and propylene glycol-containing compositions to neonates. The use of a combination of various cosolvents at a higher concentrations also causes a higher osmolality (>500 mOsm) of the product. This is of particular concern for preterm infants, who are particularly vulnerable to the adverse effects of intravenous administration of hypertonic substances, as their infusions have been associated with increased risk of intraventricular hemorrhage, hepatic necrosis and necrotizing enterocolitis. Studies have shown that hyperosmolality and metabolic acidosis, then renal dysfunction and finally acute renal failure and clinical deterioration are major effects of increasing doses and serum concentrations of propylene glycol in neonates. In addition several case reports of intoxication have pointed out the potential CNS (lethargy, coma, seizures) or local vascular affects (including hemolysis) most probably associated to higher plasma levels of propylene glycol and hyperosmolality following acute toxicity.

Propylene glycol is an example of a compound used commercially as a diluent or stabilizer in some extremely hypertonic preparations for intravenous use, including phenytoin, phenobarbital, diazepam, digoxine, and multivitamin solutions. Although it is considered to be a relatively safe substance, serum hyperosmolality and other harmful effects to newborns and infants have been reported, including secondary to transdermal absorption of propylene glycol from topical pharmaceutical preparations. In addition, the toxicity of propylene glycol to newborns and infants may be particularly acute because infants and newborns may have a delayed development of mechanisms to eliminate propylene glycol from their systems. All of these issues with propylene glycol has thus encouraged the search for alternatives to propylene glycol for use as a diluent for substances for intravenous use in neonates.

Lyophilization or freeze drying is a process in which water is removed from a product after it is frozen and placed under a vacuum, allowing the ice to change directly from solid to vapour without passing through a liquid phase.

For lyophilized drug products, conflicting requirements with respect to surface area extend beyond processing, with surface area typically identified as a CQA for the lyophilized cake. The primary goal of lyophilization is to preserve viability of the drug product while extending its shelf life, so stability is of critical concern. The links between the surface area of the cake and stability are complex but the probability of drug molecules being exposed to the cake/air interface, which is a function of surface area, is a recognized factor in degradation reactions. Surface area can also impact the likelihood of residual water getting trapped in the cake, a primary cause of compromised stability. Such correlations require detailed investigation for each formulation to determine how best to tailor surface area to support the attainment of stability targets.

Reconstitution performance of lyophilized product is also a major concern. Typically, a formulation that dissolves relatively quickly, e.g., within 10-30 sec, in a modest amount of solvent, is well suited for routine administration. Rapid dissolution reduces the risk of leaving undissolved drug in the in-line filter that precedes injection, while smaller volumes improve patient acceptability and reduce the risks associated with parenteral delivery. Here correlations with surface area may be more straightforward since it influences the contact area available to the solvent and by extension both the rate and extent of dissolution. The appearance of the cake, ‘elegance’ as it is often referred to, is a further quality metric for lyophilized cakes which preferentially take up the volume of the original sample, with minimal observable shrinkage or collapse. FDA guidance indicates a 100% visual inspection of lyophilized products, but surface area measurements can be additionally helpful in detecting and diagnosing the causes of collapse. In combination, these goals highlight the relevance of surface area data in defining the performance of the lyophilized cake and the supporting value of surface area measurements from development through scale-up to commercial production.

U.S. Patent Application Publication No. 20210085608 relates to phenobarbital sodium formulations and lyophilization. According to this publication, lyophilization may result to the stability in a phenobarbital sodium formulation. However, this publication fails to disclose or suggest the role that ethanol may play in the stability of a phenobarbital formulation.

U.S. Pat. No. 9,901,576 relates to phenobarbital formulations. However, the examples of this patent contains higher amount ethanol for the stability. According to regulatory guidelines (for example European Medicines Agency guidance and The International Council for Harmonisation of Technical Requirements for Pharmaceuticals for Human Use), the formulations having the solvent amount of over 50000 ppm are unsuitable for use in neonates.

### SUMMARY OF THE INVENTION

The present inventors have developed stable pharmaceutical compositions of the hydrolytically unstable drug phenobarbital or salts thereof. The pharmaceutical compositions of the present disclosure provides stability over a lengthy shelf life, as measured by the amount of total impurities formed over time and that does not generate any significant amount of impurities during extended storage. The present inventors surprisingly and unexpectedly discovered that freeze-drying can be carried out at or above the collapse temperature (Tc) of phenobarbital or salts thereof, while still retaining product stability, purity, activity and other important product characteristics. One of the attributes of the lyophilized product and methods of the present technology is that the product is pharmaceutically elegant and mechanically strong, and can be rapidly reconstituted without residual particles. Through their efforts, the present inventors have discovered a lyophilized product having improved morphology and pore sizes, and lyophilization methods having significant processing time and cost benefits. The present inventors also observed that the presence of higher levels of alcohol alone is not sufficient to control the level of impurities. Thus, embodiments of the present disclosure relate to the pharmaceutical compositions of phenobarbital or salts thereof and methods of preparing the same by utilizing a combination of a certain level of alcohol and lyophilization.

In the first aspect, the present disclosure relates to a method for the preparation of the lyophilized pharmaceutical composition of phenobarbital sodium, wherein the method comprises (a) dissolving phenobarbital sodium in water to obtain a pre-lyophilization solution having a concentration between 10 mg/ml to 60 mg/ml of phenobarbital sodium; and (b) lyophilizing the pre-lyophilized solution to obtain a lyophilized pharmaceutical composition.

In the second aspect, the present disclosure relates to a method for the preparation of the lyophilized pharmaceutical composition of phenobarbital sodium, wherein the method comprises (a) dissolving phenobarbital sodium in water to obtain a pre-lyophilization solution having a concentration between 10 mg/ml to 60 mg/ml of phenobarbital sodium; and (b) lyophilizing the pre-lyophilized solution to obtain a lyophilized pharmaceutical composition; and wherein step (b) comprises (i) freezing a pre-lyophilized solution of step (a) to a temperature from about 5° C. to about −45° C. to form a frozen solution; (ii) holding the frozen solution at or below −45° C., for about 200 min to about 1000 min; (iii) evacuating and ramping the frozen solution to a primary drying temperature to form a dried solution, wherein the primary drying temperature is higher than the collapse temperature of phenobarbital sodium; (iv) holding for less than about 1000 min, under a pressure of about 35 to about 120 mTorr; (v) ramping the dried solution to a secondary drying temperature from about 20° C. to about 45° C.; and (vi) holding for about 200 min to about 400 min, under a pressure of about 35 to about 60 mTorr; to form a lyophilized composition of phenobarbital or salts thereof.

In the third aspect, the present disclosure relates to a method for the preparation of the lyophilized pharmaceutical composition of phenobarbital sodium, wherein the method comprises (a) dissolving phenobarbital sodium in water to obtain a pre-lyophilization solution having a concentration between 10 mg/ml to 60 mg/ml of phenobarbital sodium; and (b) lyophilizing the pre-lyophilized solution to obtain a lyophilized pharmaceutical composition; and wherein step (b) comprises (i) freezing a pre-lyophilization solution of step (a) to a temperature of about −45° C. to form a frozen solution; (ii) holding the frozen solution at or below −45° C., for about 850 min; (iii) evacuating and ramping the frozen solution to a primary drying temperature of about 20° C. to form a dried solution; (iv) holding for less than about 30 min, under a pressure of less than about 60 mTorr; (v) ramping the dried solution to a secondary drying temperature of about 45° C.; and (vi) holding for about 330 min, under a pressure of about 50 mTorr; to form a lyophilized pharmaceutical composition of phenobarbital sodium.

In the fourth aspect, the present disclosure relates to a lyophilized pharmaceutical composition of phenobarbital sodium, prepared by a method according to the first aspect, the second aspect or the third aspect.

In the fifth aspect, the present disclosure relates to an injectable formulation comprising phenobarbital sodium, reconstituted from the lyophilized pharmaceutical composition of the fourth aspect in a sterile carrier suitable for intravenous administration.

In the sixth aspect, the present disclosure relates to a lyophilized pharmaceutical composition of phenobarbital sodium having a percent porosity in the range from about 20% to about 50%.

In seventh aspect, the lyophilized pharmaceutical composition comprises an ethanol content in the range from about 12000 ppm to about 25000 ppm.

In eighth aspect, the lyophilized pharmaceutical composition comprises total impurities in an amount of less than 0.2% following 36 months of storage at about 20° C. to about 25° C.

In ninth aspect, the lyophilized pharmaceutical composition is free of benzyl alcohol and propylene glycol.

In tenth aspect, the lyophilized pharmaceutical composition comprises lyophilized phenobarbital sodium in an amount from 10 mg/vial to 100 mg/vial. In one embodiment, the lyophilized pharmaceutical composition comprises lyophilized phenobarbital sodium in an amount of 10 mg/vial. In another embodiment, the lyophilized pharmaceutical composition comprises lyophilized phenobarbital sodium in an amount of 100 mg/vial.

In the eleventh aspect, the present disclosure relates to a lyophilized pharmaceutical composition of phenobarbital sodium having one or more of: a percent porosity in the range from about 20% to about 50%; an ethanol content in the range from about 12000 ppm to about 25000 ppm; total impurities in an amount of less than 0.2% following 36 months of storage at about 20° C. to about 25° C.; free of benzyl alcohol and propylene glycol; and lyophilized phenobarbital sodium in an amount from 10 mg/vial to 100 mg/vial.

In one aspect, the lyophilized pharmaceutical composition comprises lyophilized phenobarbital sodium in an amount of 10 mg/vial. In another aspect, the lyophilized pharmaceutical composition comprises lyophilized phenobarbital sodium in an amount of 100 mg/vial.

In another aspect, the impurities in the lyophilized pharmaceutical composition are selected from the group consisting of 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In another aspect, the lyophilized pharmaceutical composition is reconstituted with water for injection, an aqueous saline or an aqueous dextrose solution.

In another aspect, the sodium phenobarbital is present in a concentration of about 10 mg/ml to about 200 mg/ml.

In another aspect, the lyophilized pharmaceutical composition has an osmolality below 500 mOsm/kg.

In the twelfth aspect, the present disclosure relates to a lyophilized pharmaceutical composition prepared by a process comprising: (a) dissolving phenobarbital sodium in water to obtain a pre-lyophilization solution having a concentration between 10 mg/ml to 60 mg/ml of phenobarbital sodium; and (b) lyophilizing the pre-lyophilized solution of step (a).

In another aspect, the present disclosure relates to a pharmaceutical composition of phenobarbital sodium made by a method comprising reconstituting a lyophilized pharmaceutical composition.

In the thirteenth aspect, the present disclosure relates to a method for the treatment of neonatal seizure in newborn infants of 2 weeks of age or younger in need thereof, comprising administering the lyophilized pharmaceutical composition.

In another aspect, the method comprises reconstituting the lyophilized pharmaceutical composition immediately prior to the administration.

In another aspect, the lyophilized pharmaceutical composition is reconstituted with water for injection, an aqueous saline or an aqueous dextrose solution.

In the fourteenth aspect, the present disclosure relates to a pharmaceutical composition of phenobarbital or salts thereof that has been reconstituted from a lyophilized pharmaceutical composition of phenobarbital or salts thereof, wherein the amount of total impurities in the pharmaceutical composition does not exceed 0.2% following 12 hours of storage at 20-25° C. or 36 hours of storage at 2-8° C.

In the fifteenth aspect, the present disclosure relates to a pharmaceutical composition of phenobarbital or salts thereof, wherein the pharmaceutical composition has an alcohol content in the range from about 5000 ppm to 66000 ppm, alternatively from about 5000 ppm to 70000 ppm, or from about 12000 ppm to about 25000 ppm.

In the sixteenth aspect, the present disclosure relates to a lyophilized pharmaceutical composition of phenobarbital or salts thereof, wherein the composition has an alcohol content in the range from about 5000 ppm to about 66000 ppm, alternatively from about 5000 ppm to about 70000 ppm, or from about 12000 ppm to about 25000 ppm.

In the seventeenth aspect, the present disclosure relates to a pharmaceutical composition of phenobarbital or salts thereof that has been reconstituted from a lyophilized pharmaceutical composition of phenobarbital or salts thereof, wherein the pharmaceutical composition has an alcohol content in the range from about 5000 ppm to about 66000 ppm, alternatively from about 5000 ppm to about 70000 ppm, or from about 12000 ppm to about 25000 ppm.

In the eighteenth aspect, the present disclosure relates to a process of preparing the lyophilized pharmaceutical composition of phenobarbital or salts thereof having an alcohol content in the range from about 5000 ppm to about 66000 ppm or about 70000 ppm, or alternatively from about 12000 ppm to about 25000 ppm, wherein the process comprises dissolving phenobarbital or salts thereof in water to obtain an aqueous solution of phenobarbital or salts thereof in a concentration of 10-200 mg/ml and lyophilizing the aqueous solution to obtain lyophilized pharmaceutical composition of phenobarbital or salts thereof.

In the nineteenth aspect, the present disclosure relates to a process of preparing the pharmaceutical composition of phenobarbital or salts thereof that has been reconstituted from a lyophilized pharmaceutical composition of phenobarbital or salts thereof, wherein the pharmaceutical composition has an alcohol content in the range from about 5000 ppm to about 66000 ppm or about 70000 ppm, or alternatively from about 12000 ppm to about 25000 ppm, the process comprises dissolving phenobarbital or salts thereof in water to obtain an aqueous solution having a concentration 10-200 mg/ml; lyophilizing the aqueous solution to obtain lyophilized pharmaceutical composition of phenobarbital or salts thereof; and reconstituting the lyophilized pharmaceutical composition to obtain the pharmaceutical composition of phenobarbital or salts thereof.

In the twentieth aspect, the present disclosure relates to a lyophilized pharmaceutical composition of phenobarbital or salts thereof having an alcohol content in the range from about 5000 ppm to about 66000 ppm, alternatively from about 5000 ppm to about 70000 ppm, or from about 12000 ppm to about 25000 ppm, wherein the lyophilized pharmaceutical composition is obtained by a process comprising: dissolving phenobarbital or salts thereof in water to obtain an aqueous solution having a concentration 10-200 mg/ml and lyophilizing the aqueous solution to obtain lyophilized pharmaceutical composition of phenobarbital or salts thereof.

In the twenty-first aspect, the present disclosure relates to a pharmaceutical composition of phenobarbital or salts thereof that has been reconstituted from a lyophilized pharmaceutical composition of phenobarbital or salts thereof having an alcohol content in the range from about 5000 ppm to about 66000 ppm, alternatively from about 5000 ppm to about 70000 ppm, or from about 12000 ppm to about 25000 ppm, wherein the pharmaceutical composition is obtained by a process comprising: dissolving phenobarbital or salts thereof in water to obtain an aqueous solution having a concentration 10-200 mg/ml; lyophilizing the aqueous solution to obtain lyophilized pharmaceutical composition of phenobarbital or salts thereof, and reconstituting the lyophilized pharmaceutical composition to obtain the pharmaceutical composition of phenobarbital or salts thereof.

In the twenty-second aspect, the present disclosure relates to a lyophilized pharmaceutical composition comprising phenobarbital sodium and ethanol, wherein ethanol is present in an amount sufficient to inhibit degradation of phenobarbital sodium, such that the amount of total impurities present following 36 months of storage at 20-25° C. does not exceed 0.2%.

In the twenty-third aspect, the present disclosure relates to an aqueous solution for injection comprising phenobarbital sodium and ethanol, wherein ethanol is present in an amount sufficient to inhibit degradation of phenobarbital sodium, such that the amount of total impurities present following 12 hours of storage at 20-25° C. or following 36 hours of storage at 2-8° C. does not exceed 0.2%.

In the twenty-fourth aspect, the present disclosure relates to a process for the preparation of the pharmaceutical composition of phenobarbital or salts thereof, wherein the process comprises dissolving phenobarbital or salt thereof in water to obtain an aqueous solution having a concentration 10-200 mg/ml and lyophilizing the aqueous solution to obtain lyophilized pharmaceutical composition, wherein phenobarbital or salts thereof has an alcohol content in the range from about 5000 ppm to about 66000 ppm, alternatively from about 5000 ppm to about 70000 ppm, or from about 12000 ppm to about 25000 ppm.

In another aspect, the present disclosure relates to a lyophilized pharmaceutical composition of phenobarbital or salts thereof prepared by the methods described herein.

In another aspect, the present disclosure relates to a lyophilized pharmaceutical composition of phenobarbital sodium, wherein the composition has an ethanol content in the range from about 12000 ppm to about 25000 ppm; wherein the composition is stable up to 36 months of storage at 20-25° C. such that the amount of total impurities present following 36 months of storage at 20-25° C. does not exceed 0.2%; and wherein the composition is free of benzyl alcohol and propylene glycol.

In another aspect, the present disclosure relates to an aqueous solution for injection of phenobarbital sodium, wherein the aqueous solution has an ethanol content in the range from about 12000 ppm to about 25000 ppm; wherein the aqueous solution is stable up to 12 hours of storage at 20-25° C. or 36 hours of storage at 2-8° C. such that the amount of total impurities present following 12 hours of storage at 20-25° C. or following 36 hours of storage at 2-8° C. does not exceed 0.2%; wherein the aqueous solution is reconstituted from the lyophilized pharmaceutical composition of phenobarbital sodium; and wherein the aqueous solution is free of benzyl alcohol and propylene glycol.

In another aspect, the present disclosure relates to a lyophilized pharmaceutical composition of phenobarbital or salts thereof, wherein the amount of alcohol in the lyophilized pharmaceutical composition following 36 months of storage at 20-25° C., is in the range from about 5000 ppm to about 66000 ppm or alternatively from about 5000 ppm to about 70000 ppm.

In another aspect, the present disclosure relates to a pharmaceutical composition of phenobarbital or salts thereof that has been reconstituted from a lyophilized pharmaceutical composition of phenobarbital or salts thereof, wherein the amount of alcohol in the pharmaceutical composition following 12 hours of storage at 20-25° C. or 36 hours of storage at 2-8° C., is in the range from about 5000 ppm to 66000 ppm, alternatively from about 5000 ppm to about 70000 ppm, or from about 12000 ppm to about 25000 ppm.

In another aspect, the present disclosure relates to a method of preventing degradation of phenobarbital or salts thereof, wherein the method comprises dissolving phenobarbital or salts thereof in water to obtain an aqueous solution having a concentration 10-200 mg/ml; and lyophilizing the aqueous solution to obtain lyophilized pharmaceutical composition of phenobarbital or salts thereof, wherein phenobarbital or salts thereof has an alcohol content in the range from about 5000 ppm to about 66000 ppm, alternatively from about 5000 ppm to about 70000 ppm, or from about 12000 ppm to about 25000 ppm.

### BRIEF DESCRIPTION OF THE DRAWINGS

FIG. 1 is a photograph of vials containing lyophilized phenobarbital sodium prepared from 2.5 mg/mL, 5 mg/mL, 10 mg/mL and 25 mg/mL of pre-lyophilization phenobarbital sodium solution.

FIG. 2 illustrates Scanning Electron Microscopy (SEM) images showing increased surface area per gram of lyophilized phenobarbital sodium powder with increasing dilution of the lyophilization solution.

### DETAILED DESCRIPTION OF THE INVENTION

### Definitions

The “phenobarbital or salts thereof” that may be used according to the present disclosure may be phenobarbital base or a phenobarbital salt. The particular salt form of phenobarbital is not particularly limited, and in non-limiting examples, may be, for example, phenobarbital sodium, phenobarbital potassium, phenobarbital benzathine, phenobarbital betaine, or phenobarbital choline. Preferred embodiments utilize a phenobarbital sodium salt.

The term “reconstitution” as used herein, includes the addition of vehicle/diluent in to the lyophilized phenobarbital lyophilized phenobarbital. In preferred embodiments, the vehicle/diluent is water for injection, an aqueous saline solution or an aqueous dextrose solution. In additional preferred embodiments, the vehicle/diluent is water for injection or a 0.9% aqueous saline solution.

The terms “about” as used herein refers to as modifying a term or value such that it is not an absolute. Such terms will be defined by the circumstances and the terms that they modify as those terms are understood by those of skill in the art. This includes, at very least, the degree of expected experimental error, technique error and instrument error for a given technique used to measure a value. The term “about” when used in the present application preceding a number and referring to it, is meant to designate any value which lies within the range of 10%.

The term “total impurities” as used herein, includes known and unknown impurities, either present from the active pharmaceutical ingredient (API) or generated by the degradation of phenobarbital or salts thereof during the manufacturing or stability of the pharmaceutical compositions of the present disclosures. These total impurities includes but not limited to 2-phenyl-2-ethyl acetyl urea, 2-phenyl-2-ethyl-malonamide, α-phenylbutyrylguanidine, 2-phenylbutyric acid and 5-methyl-5-phenylbarbituric acid, and can be represented by following structural formulas:

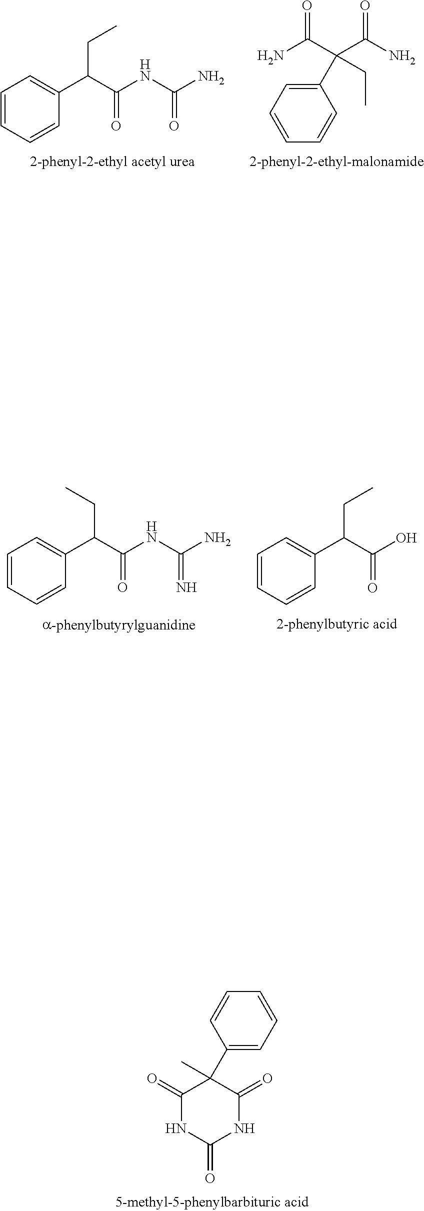 [SMILES: CCC(C(=O)NC(N)=O)c1ccccc1 ; CCC(C(N)=O)(C(N)=O)c1ccccc1 ; CCC(C(=O)NC(=N)N)c1ccccc1 ; CCC(C(=O)O)c1ccccc1 ; CC1(c2ccccc2)C(=O)NC(=O)NC1=O]

The term “correctable abnormalities” as used herein is defined as any abnormality which can be corrected by medication. The correctable abnormalities include hypoglycemia or hypocalcemia.

The term “C₁-C₃ alcohol” as used herein means an alkanol having 1-3 carbon atoms which include methanol, ethanol, 1-propanol, or isopropyl alcohol.

The term “collapse temperature” (Tc) as used herein refers to the temperature at which the phenobarbital or salts thereof softens to the point of not being able to support its own structure.

The collapse temperature can be measured using methods known in the art, including Freeze-drying microscopy. The principal information obtained from FDM analysis is the collapse (Tc) or eutectic (Teu) point of the formulation.

In the first aspect, the present disclosure relates to a pharmaceutical composition of phenobarbital or salts thereof that has been reconstituted from a lyophilized pharmaceutical composition of phenobarbital or salts thereof.

In one embodiment, the pharmaceutical composition of phenobarbital or salts thereof is an aqueous solution for injection.

In one embodiment, the amount of total impurities in the pharmaceutical composition does not exceed 0.2% following 12 hours of storage at 20-25° C. or 36 hours of storage at 2-8° C.

In one embodiment, the present disclosure relates to a pharmaceutical composition of phenobarbital or salts thereof that has been reconstituted from a lyophilized pharmaceutical composition of phenobarbital or salts thereof, wherein the amount of total impurities in the pharmaceutical composition does not exceed 0.2% following 12 hours of storage at 20-25° C. or 36 hours of storage at 2-8° C. Alternatively, the total impurities does not exceed 0.15% following 12 hours of storage at 20-25° C. or 36 hours of storage at 2-8° C., alternatively, the total impurities does not exceed 0.1% following 12 hours of storage at 20-25° C. or 36 hours of storage at 2-8° C., alternatively, the total impurities does not exceed 0.05% following 12 hours of storage at 20-25° C. or 36 hours of storage at 2-8° C.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the lyophilized pharmaceutical composition of phenobarbital or salts thereof is reconstituted with water for injection, an aqueous saline or an aqueous dextrose solution. In one embodiment, the lyophilized pharmaceutical composition of phenobarbital or salts thereof is reconstituted with 0.9% of aqueous saline.

In one embodiment, phenobarbital or salts thereof is present in a concentration of 10-200 mg/ml. In one embodiment, phenobarbital or salts thereof is present in a concentration of 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml and 200 mg/ml.

In one embodiment, phenobarbital or salts thereof is phenobarbital base or phenobarbital sodium, preferably phenobarbital sodium.

In one embodiment, the pharmaceutical composition has an alcohol content in the range from about 5000 ppm to about 66000 ppm or alternatively from about 5000 ppm to about 70000 ppm. In one embodiment, the alcohol content is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, the osmolality of said pharmaceutical composition is below 500 mOsm/kg.

In one embodiment, the osmolality of said pharmaceutical composition is about 300-400 mOsm/kg.

In one embodiment, the pharmaceutical composition is free of benzyl alcohol.

In one embodiment, the pharmaceutical composition is also free of propylene glycol.

In a preferred embodiment, the pharmaceutical composition is free of benzyl alcohol and propylene glycol.

In another aspect, the present disclosure relates to a method for the treatment of neonatal seizure in newborn infants in need thereof, comprising administering the pharmaceutical composition of phenobarbital or salts thereof that has been reconstituted from a lyophilized pharmaceutical composition of phenobarbital or salts thereof, wherein the amount of total impurities in the pharmaceutical composition does not exceed 0.2% following 12 hours of storage at 20-25° C. or 36 hours of storage at 2-8° C. Alternatively, the total impurities does not exceed 0.15% following 12 hours of storage at 20-25° C. or 36 hours of storage at 2-8° C., alternatively, the total impurities does not exceed 0.1% following 12 hours of storage at 20-25° C. or 36 hours of storage at 2-8° C., alternatively, the total impurities does not exceed 0.05% following 12 hours of storage at 20-25° C. or 36 hours of storage at 2-8° C.

In one embodiment, the newborn infants is of 2 weeks of age or younger.

In one embodiment, the present disclosure relates to a method for the treatment of neonatal seizure in newborn infants of 2 weeks of age or younger in need thereof, comprising administering the pharmaceutical composition of phenobarbital or salts thereof that has been reconstituted from a lyophilized phenobarbital or salts thereof, wherein the amount of total impurities in the pharmaceutical composition does not exceed 0.2% following 12 hours of storage at 20-25° C. or 36 hours of storage at 2-8° C. Alternatively, the total impurities does not exceed 0.15% following 12 hours of storage at 20-25° C. or 36 hours of storage at 2-8° C., alternatively, the total impurities does not exceed 0.1% following 12 hours of storage at 20-25° C. or 36 hours of storage at 2-8° C., alternatively, the total impurities does not exceed 0.05% following 12 hours of storage at 20-25° C. or 36 hours of storage at 2-8° C.

In one embodiment, the method comprises administering the pharmaceutical composition to neonates in whom correctable abnormalities have been excluded or corrected. In one embodiment, the said correctable abnormalities are hypoglycemia or hypocalcemia.

In one embodiment, wherein the pharmaceutical composition is administered intravenously by infusion at a dose of 20 mg/kg over a course of 15 minutes. In one embodiment, the method comprises administration of the pharmaceutical composition at an initial loading dose of 20 mg/kg over a course of 15 minutes and measuring the electrographic seizures, wherein if the electrographic seizures persist or recur after completion of the initial loading dose, a second dose 20 mg/kg is administered over the subsequent 15 minutes for a total loading dose of 40 mg/kg. Electrographic seizures can be measured by any means and instruments known in the art like electroencephalogram (EEG) or using 2-channel EEG with amplitude-integrated EEG.

In one embodiment, wherein the method for the treatment of neonatal seizure in newborn infants comprising administering the pharmaceutical composition of the first aspect.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the pharmaceutical composition of phenobarbital or salts thereof is an aqueous solution for injection.

In one embodiment, the lyophilized pharmaceutical composition of phenobarbital or salts thereof is reconstituted with water for injection, an aqueous saline or an aqueous dextrose solution. In one embodiment, the lyophilized pharmaceutical composition of phenobarbital or salts thereof is reconstituted with 0.9% of aqueous saline.

In one embodiment, phenobarbital or salts thereof is present in a concentration of 10-200 mg/ml. In one embodiment, phenobarbital or salts thereof is present in a concentration of 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml and 200 mg/ml.

In one embodiment, phenobarbital or salts thereof is phenobarbital base or phenobarbital sodium, preferably phenobarbital sodium.

In one embodiment, the pharmaceutical composition has an alcohol content in the range from about 5000 ppm to about 66000 ppm or alternatively from about 5000 ppm to about 70000 ppm. In one embodiment, the alcohol content is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, the osmolality of said pharmaceutical composition is below 500 mOsm/kg.

In one embodiment, the osmolality of said pharmaceutical composition is about 300-400 mOsm/kg.

In one embodiment, the pharmaceutical composition is free of benzyl alcohol.

In one embodiment, the pharmaceutical composition is also free of propylene glycol.

In a preferred embodiment, the pharmaceutical composition is free of benzyl alcohol and propylene glycol.

In the second aspect, the present disclosure relates to a pharmaceutical composition of phenobarbital or salts thereof, wherein the pharmaceutical composition has an alcohol content in the range from about 5000 ppm to 66000 ppm or alternatively from about 5000 ppm to about 70000 ppm.

In one embodiment, the pharmaceutical composition has an alcohol content in the range from about 5000 ppm to about 66000 ppm or alternatively from about 5000 ppm to about 70000 ppm. In one embodiment, the alcohol content is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In another embodiment of the second aspect, the pharmaceutical composition is a lyophilized pharmaceutical composition of phenobarbital or salts thereof.

In one embodiment, the lyophilized pharmaceutical composition is stable up to 36 months of storage at 20-25° C.

In one embodiment, the amount of total impurities present in the lyophilized pharmaceutical composition following 36 months of storage at 20-25° C. does not exceed 0.5%. In one embodiment, the amount of total impurities present in the lyophilized pharmaceutical composition following 36 months of storage at 20-25° C. does not exceed 0.2%.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In another embodiment of the second aspect, the pharmaceutical composition is an aqueous solution for injection of phenobarbital or salts thereof.

In one embodiment, the aqueous solution is reconstituted from the lyophilized pharmaceutical composition of phenobarbital or salts thereof. In one embodiment, the lyophilized pharmaceutical composition is reconstituted with water for injection, an aqueous saline or an aqueous dextrose solution. In another embodiment, the lyophilized pharmaceutical composition is reconstituted with 0.9% of aqueous saline.

In one embodiment, the aqueous solution comprise phenobarbital or salts thereof in a concentration of 10-200 mg/ml. In one embodiment, phenobarbital or salts thereof is present in a concentration of 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml and 200 mg/ml.

In one embodiment, the aqueous solution is stable up to 12 hours of storage at 20-25° C.

In one embodiment, the amount of total impurities present in the aqueous solution following 12 hours of storage at 20-25° C. does not exceed 0.5%. In one embodiment, the amount of total impurities present in the aqueous solution following 12 hours of storage at 20-25° C. does not exceed 0.2%.

In one embodiment, the aqueous solution is stable up to 36 hours of storage at 2-8° C.

In one embodiment, the amount of total impurities present in the aqueous solution following 36 hours of storage at 2-8° C. does not exceed 0.5%. In one embodiment, the amount of total impurities present in the aqueous solution following 36 hours of storage at 2-8° C. does not exceed 0.2%.

Alternatively, the total impurities does not exceed 0.15% following 12 hours of storage at 20-25° C. or 36 hours of storage at 2-8° C., alternatively, the total impurities does not exceed 0.1% following 12 hours of storage at 20-25° C. or 36 hours of storage at 2-8° C., alternatively, the total impurities does not exceed 0.05% following 12 hours of storage at 20-25° C. or 36 hours of storage at 2-8° C.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the osmolality of said composition is below 500 mOsm/kg. In one embodiment, the osmolality of said aqueous solution is about 300-400 mOsm/kg.

In one embodiment, the pharmaceutical composition is free of benzyl alcohol.

In one embodiment, the pharmaceutical composition is also free of propylene glycol.

In a preferred embodiment, the pharmaceutical composition is free of benzyl alcohol and propylene glycol.

In another aspect, the present disclosure relates to a method for the treatment of neonatal seizure in newborn infants in need thereof, comprising administering the pharmaceutical composition of phenobarbital or salts thereof, wherein the pharmaceutical composition has an alcohol content in the range from about 5000 ppm to about 66000 ppm or alternatively from about 5000 ppm to about 70000 ppm.

In one embodiment, the newborn infants is 2 weeks of age or younger.

In one embodiment, the present disclosure relates to a method for the treatment of neonatal seizure in newborn infants of 2 weeks of age or younger in need thereof, comprising administering the pharmaceutical composition of phenobarbital or salts thereof, wherein the pharmaceutical composition has an alcohol content in the range from about 5000 ppm to about 66000 ppm or alternatively from about 5000 ppm to about 70000 ppm.

In one embodiment, the method comprises administering the pharmaceutical composition to neonates in whom correctable abnormalities have been excluded or corrected. In one embodiment, the said correctable abnormalities are hypoglycemia or hypocalcemia.

In one embodiment, wherein the pharmaceutical composition of the second aspect is administered intravenously by infusion at a dose of 20 mg/kg over a course of 15 minutes.

In one embodiment, wherein the method comprises administration of the pharmaceutical composition at an initial loading dose of 20 mg/kg over a course of 15 minutes and measuring the electrographic seizures, wherein if the electrographic seizures persist or recur after completion of the initial loading dose, a second dose 20 mg/kg is administered over the subsequent 15 minutes for a total loading dose of 40 mg/kg.

In one embodiment, wherein the method for the treatment of neonatal seizure in newborn infants comprising administering the pharmaceutical composition of the second aspect.

In one embodiment, the pharmaceutical composition has an alcohol content in the range from about 5000 ppm to about 66000 ppm or alternatively from about 5000 ppm to about 70000 ppm. In one embodiment, the alcohol content is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In one embodiment, the pharmaceutical composition is free of benzyl alcohol.

In one embodiment, the pharmaceutical composition is also free of propylene glycol.

In a preferred embodiment, the pharmaceutical composition is free of benzyl alcohol and propylene glycol.

In another embodiment, the pharmaceutical composition is a lyophilized pharmaceutical composition of phenobarbital or salts thereof.

In one embodiment, the lyophilized pharmaceutical composition is stable up to 36 months of storage at 20-25° C.

In one embodiment, the amount of total impurities present in the lyophilized pharmaceutical composition following 36 months of storage at 20-25° C. does not exceed 0.5%. In one embodiment, the amount of total impurities present in the lyophilized pharmaceutical composition following 36 months of storage at 20-25° C. does not exceed 0.2%.

In another embodiment, the pharmaceutical composition is an aqueous solution for injection of phenobarbital or salts thereof.

In one embodiment, the aqueous solution is reconstituted from the lyophilized pharmaceutical composition of phenobarbital or salts thereof. In one embodiment, the lyophilized pharmaceutical composition is reconstituted with water for injection, an aqueous saline or an aqueous dextrose solution. In another embodiment, the lyophilized pharmaceutical composition is reconstituted with 0.9% of aqueous saline.

In one embodiment, the aqueous solution comprise phenobarbital or salts thereof in a concentration of 10-200 mg/ml. In one embodiment, phenobarbital or salts thereof is present in a concentration of 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml and 200 mg/ml.

In one embodiment, the aqueous solution is stable up to 12 hours of storage at 20-25° C.

In one embodiment, the amount of total impurities present in the aqueous solution following 12 hours of storage at 20-25° C. does not exceed 0.5%. In one embodiment, the amount of total impurities present in the aqueous solution following 12 hours of storage at 20-25° C. does not exceed 0.2%.

In one embodiment, the aqueous solution is stable up to 36 hours of storage at 2-8° C.

In one embodiment, the amount of total impurities present in the aqueous solution following 36 hours of storage at 2-8° C. does not exceed 0.5%. In one embodiment, the amount of total impurities present in the aqueous solution following 36 hours of storage at 2-8° C. does not exceed 0.2%.

Alternatively, the total impurities does not exceed 0.15% following 12 hours of storage at 20-25° C. or 36 hours of storage at 2-8° C., alternatively, the total impurities does not exceed 0.1% following 12 hours of storage at 20-25° C. or 36 hours of storage at 2-8° C., alternatively, the total impurities does not exceed 0.05% following 12 hours of storage at 20-25° C. or 36 hours of storage at 2-8° C.

In one embodiment, the osmolality of said composition is below 500 mOsm/kg. In one embodiment, the osmolality of said aqueous solution is about 300-400 mOsm/kg. In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In the third aspect, the present disclosure relates to a lyophilized pharmaceutical composition of phenobarbital or salts thereof, wherein the composition has an alcohol content in the range from about 5000 ppm to about 66000 ppm or alternatively from about 5000 ppm to about 70000 ppm.

In one embodiment, the lyophilized pharmaceutical composition has an alcohol content in the range from about 5000 ppm to about 66000 ppm or alternatively from about 5000 ppm to about 70000 ppm. In one embodiment, the alcohol content is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In one embodiment, the lyophilized pharmaceutical composition is stable up to 36 months of storage at 20-25° C.

In one embodiment, the amount of total impurities present in the lyophilized pharmaceutical composition following 36 months of storage at 20-25° C. does not exceed 0.5%. In one embodiment, the amount of total impurities present in the lyophilized pharmaceutical composition following 36 months of storage at 20-25° C. does not exceed 0.2%.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the lyophilized pharmaceutical composition is free of benzyl alcohol.

In one embodiment, the lyophilized pharmaceutical composition is also free of propylene glycol.

In a preferred embodiment, the lyophilized pharmaceutical composition is free of benzyl alcohol and propylene glycol.

In another aspect, the present disclosure relates to a method for the treatment of neonatal seizure in newborn infants in need thereof, comprising administering the lyophilized pharmaceutical composition of phenobarbital or salts thereof, and wherein the lyophilized pharmaceutical composition has an alcohol content in the range from about 5000 ppm to about 66000 ppm.

In one embodiment, the newborn infants is of 2 weeks of age or younger.

In one embodiment, the present disclosure relates to a method for the treatment of neonatal seizure in newborn infants of 2 weeks of age or younger in need thereof, comprising administering the lyophilized pharmaceutical composition of phenobarbital or salts thereof, and wherein the lyophilized pharmaceutical composition has an alcohol content in the range from about 5000 ppm to about 66000 ppm.

In one embodiment, the method comprises reconstituting the lyophilized pharmaceutical composition of phenobarbital or salts thereof immediately prior to the administration.

In one embodiment, the lyophilized pharmaceutical composition is reconstituted with water for injection, an aqueous saline or an aqueous dextrose solution to obtain the aqueous solution for injection of phenobarbital or salts thereof.

In one embodiment, the method comprises administering the aqueous solution to neonates in whom correctable abnormalities have been excluded or corrected. In one embodiment, the said correctable abnormalities are hypoglycemia or hypocalcemia.

In one embodiment, wherein the aqueous solution is administered intravenously by infusion at a dose of 20 mg/kg over a course of 15 minutes.

In one embodiment, wherein the method comprises administration of the aqueous solution at an initial loading dose of 20 mg/kg over a course of 15 minutes and measuring the electrographic seizures, wherein if the electrographic seizures persist or recur after completion of the initial loading dose, a second dose 20 mg/kg is administered over the subsequent 15 minutes for a total loading dose of 40 mg/kg.

In one embodiment, wherein the method for the treatment of neonatal seizure in newborn infants comprising administering the lyophilized pharmaceutical composition of the third aspect.

In one embodiment, the lyophilized pharmaceutical composition has an alcohol content in the range from about 5000 ppm to about 66000 ppm or alternatively from about 5000 ppm to about 70000 ppm. In one embodiment, the alcohol content is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In one embodiment, the lyophilized pharmaceutical composition is stable up to 36 months of storage at 20-25° C.

In one embodiment, the amount of total impurities present in the lyophilized pharmaceutical composition following 36 months of storage at 20-25° C. does not exceed 0.5%. In one embodiment, the amount of total impurities present in the lyophilized pharmaceutical composition following 36 months of storage at 20-25° C. does not exceed 0.2%.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the lyophilized pharmaceutical composition is free of benzyl alcohol.

In one embodiment, the lyophilized pharmaceutical composition is also free of propylene glycol.

In a preferred embodiment, the lyophilized pharmaceutical composition is free of benzyl alcohol and propylene glycol.

In the fourth aspect, the present disclosure relates to a pharmaceutical composition of phenobarbital or salts thereof that has been reconstituted from a lyophilized pharmaceutical composition of phenobarbital or salts thereof, wherein the pharmaceutical composition has an alcohol content in the range from about 5000 ppm to about 66000 ppm or alternatively from about 5000 ppm to about 70000 ppm.

In one embodiment, the pharmaceutical composition has an alcohol content in the range from about 5000 ppm to about 66000 ppm or alternatively from about 5000 ppm to about 70000 ppm. In one embodiment, the alcohol content is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In one embodiment, the lyophilized pharmaceutical composition is reconstituted with water for injection, an aqueous saline or an aqueous dextrose solution. In one embodiment, the lyophilized pharmaceutical composition is reconstituted with 0.9% aqueous saline.

In one embodiment, the pharmaceutical composition is an aqueous solution for injection of phenobarbital or salts thereof.

In one embodiment, the pharmaceutical composition comprise phenobarbital or salts thereof in a concentration of 10-200 mg/ml. In one embodiment, phenobarbital or salts thereof is present in a concentration of 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml and 200 mg/ml.

In one embodiment, the pharmaceutical composition is stable up to 12 hours of storage at 20-25° C.

In one embodiment, the amount of total impurities present in the pharmaceutical composition following 12 hours of storage at 20-25° C. does not exceed 0.5%. In one embodiment, the amount of total impurities present in the pharmaceutical composition following 12 hours of storage at 20-25° C. does not exceed 0.2%.

In one embodiment, the pharmaceutical composition is stable up to 36 hours of storage at 2-8° C.

In one embodiment, the amount of total impurities present in the pharmaceutical composition following 36 hours of storage at 2-8° C. does not exceed 0.5%. In one embodiment, the amount of total impurities present in the pharmaceutical composition following 36 hours of storage at 2-8° C. does not exceed 0.2%.

Alternatively, the total impurities does not exceed 0.15% following 12 hours of storage at 20-25° C. or 36 hours of storage at 2-8° C., alternatively, the total impurities does not exceed 0.1% following 12 hours of storage at 20-25° C. or 36 hours of storage at 2-8° C., alternatively, the total impurities does not exceed 0.05% following 12 hours of storage at 20-25° C. or 36 hours of storage at 2-8° C.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the osmolality of said pharmaceutical composition is below 500 mOsm/kg.

In one embodiment, the osmolality of said aqueous solution is about 300-400 mOsm/kg.

In one embodiment, the pharmaceutical composition is free of benzyl alcohol.

In one embodiment, the pharmaceutical composition is also free of propylene glycol.

In a preferred embodiment, the pharmaceutical composition is free of benzyl alcohol and propylene glycol.

In another aspect, the present disclosure relates to a method for the treatment of neonatal seizure in newborn infants in need thereof, comprising administering the pharmaceutical composition of phenobarbital or salts thereof that has been reconstituted from a lyophilized pharmaceutical composition of phenobarbital or salts thereof, and wherein the pharmaceutical composition has an alcohol content in the range from about 5000 ppm to about 66000 ppm or alternatively from about 5000 ppm to about 70000 ppm.

In one embodiment, the newborn infants is of 2 weeks of age or younger.

In one embodiment, the present disclosure relates to a method for the treatment of neonatal seizure in newborn infants of 2 weeks of age or younger in need thereof, comprising administering the pharmaceutical composition of phenobarbital or salts thereof that has been reconstituted from a lyophilized pharmaceutical composition of phenobarbital or salts thereof, and wherein the pharmaceutical composition has an alcohol content in the range from about 5000 ppm to about 66000 ppm or alternatively from about 5000 ppm to about 70000 ppm.

In one embodiment, the method comprises administering the pharmaceutical composition to neonates in whom correctable abnormalities have been excluded or corrected. In one embodiment, the said correctable abnormalities are hypoglycemia or hypocalcemia.

In one embodiment, wherein the pharmaceutical composition is administered intravenously by infusion at a dose of 20 mg/kg over a course of 15 minutes.

In one embodiment, wherein the method comprises administration of the pharmaceutical composition at an initial loading dose of 20 mg/kg over a course of 15 minutes and measuring the electrographic seizures, wherein if the electrographic seizures persist or recur after completion of the initial loading dose, a second dose 20 mg/kg is administered over the subsequent 15 minutes for a total loading dose of 40 mg/kg.

In one embodiment, wherein the method for the treatment of neonatal seizure in newborn infants comprising administering the pharmaceutical composition of the fourth aspect.

In one embodiment, the pharmaceutical composition has an alcohol content in the range from about 5000 ppm to about 66000 ppm or alternatively from about 5000 ppm to about 70000 ppm. In one embodiment, the alcohol content is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In one embodiment, the lyophilized pharmaceutical composition is reconstituted with water for injection, an aqueous saline or an aqueous dextrose solution. In one embodiment, the lyophilized pharmaceutical composition is reconstituted with 0.9% aqueous saline.

In one embodiment, the pharmaceutical composition is an aqueous solution for injection of phenobarbital or salts thereof.

In one embodiment, the pharmaceutical composition comprise phenobarbital or salts thereof in a concentration of 10-200 mg/ml. In one embodiment, phenobarbital or salts thereof is present in a concentration of 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml and 200 mg/ml.

In one embodiment, the pharmaceutical composition is stable up to 12 hours of storage at 20-25° C.

In one embodiment, the amount of total impurities present in the pharmaceutical composition following 12 hours of storage at 20-25° C. does not exceed 0.5%. In one embodiment, the amount of total impurities present in the pharmaceutical composition following 12 hours of storage at 20-25° C. does not exceed 0.2%.

In one embodiment, the pharmaceutical composition is stable up to 36 hours of storage at 2-8° C.

In one embodiment, the amount of total impurities present in the pharmaceutical composition following 36 hours of storage at 2-8° C. does not exceed 0.5%. In one embodiment, the amount of total impurities present in the pharmaceutical composition following 36 hours of storage at 2-8° C. does not exceed 0.2%.

Alternatively, the total impurities does not exceed 0.15% following 12 hours of storage at 20-25° C. or 36 hours of storage at 2-8° C., alternatively, the total impurities does not exceed 0.1% following 12 hours of storage at 20-25° C. or 36 hours of storage at 2-8° C., alternatively, the total impurities does not exceed 0.05% following 12 hours of storage at 20-25° C. or 36 hours of storage at 2-8° C.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the osmolality of the pharmaceutical composition is below 500 mOsm/kg.

In one embodiment, the osmolality of the pharmaceutical composition is about 300-400 mOsm/kg.

In one embodiment, the pharmaceutical composition is free of benzyl alcohol.

In one embodiment, the pharmaceutical composition is also free of propylene glycol.

In a preferred embodiment, the pharmaceutical composition is free of benzyl alcohol and propylene glycol.

In the fifth aspect, the present disclosure relates to a process of preparing the lyophilized pharmaceutical composition of phenobarbital or salts thereof having an alcohol content in the range from about 5000 ppm to about 66000 ppm or alternatively from about 5000 ppm to about 70000 ppm, wherein the process comprises dissolving phenobarbital or salts thereof in water to obtain an aqueous solution of phenobarbital or salts thereof in a concentration of 10-200 mg/ml and lyophilizing the aqueous solution to obtain lyophilized pharmaceutical composition of phenobarbital or salts thereof.

In one embodiment, the process comprises measuring the alcohol content of an aqueous solution of phenobarbital or salts thereof. In one embodiment, if the alcohol content of the aqueous solution is below 5000 ppm then the process further comprises a step of adding an alcohol in a quantity that is sufficient to achieve the alcohol content of at least about 5000 ppm. In one embodiment, the process comprises measuring the alcohol content of the lyophilized pharmaceutical composition of phenobarbital or salts thereof. In one embodiment, the process may comprises repeating steps of lyophilization to achieve the alcohol content not more than about 66000 ppm or about 70000 ppm. In one embodiment, if the alcohol content is above 66000 ppm or 70000 ppm (whichever is desired), the process further comprises repeating the lyophilization step until the alcohol content of the lyophilized pharmaceutical composition is not more than about 66000 ppm or not more than about 70000 ppm. It is understood that even a single additional cycle of lyophilization would be sufficient or it has to be repeated n number of times to obtain the desired alcohol level.

In another aspect, the present disclosure relates to a process of preparing the lyophilized pharmaceutical composition of phenobarbital or salts thereof having an alcohol content in the range from about 5000 ppm to about 66000 ppm or alternatively from about 5000 ppm to about 70000 ppm, wherein the process comprises dissolving phenobarbital or salts thereof in water to obtain an aqueous solution having a concentration 10-200 mg/ml; measuring the alcohol content of aqueous solution; if the alcohol content is below 5000 ppm, adding an alcohol to achieve the alcohol content of at least about 5000 ppm; lyophilizing the aqueous solution to obtain lyophilized pharmaceutical composition; measuring the alcohol content of the lyophilized pharmaceutical composition; if the alcohol content is above 66000 ppm or above about 70000 ppm (whichever is desired), repeating the lyophilization step multiple times till the alcohol content of the lyophilized pharmaceutical composition is not more than about 66000 ppm or is not more than about 70000 ppm.

In one embodiment, the process comprises addition of a pH modifier to the aqueous solution of phenobarbital or salts thereof to achieve a pH in a range of 8-12, preferably in a range of 9-11, more preferably in a range of 9-10.5.

In one embodiment, the pH modifier is selected from HCl and/or NaOH. In one embodiment, the pH modifier is aqueous HCl solution.

In one embodiment, the lyophilized pharmaceutical composition is stable up to 36 months of storage at 20-25° C.

In one embodiment, the lyophilized pharmaceutical composition has an alcohol content of at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In one embodiment, the lyophilized pharmaceutical composition is stable up to 36 months of storage at 20-25° C.

In one embodiment, the amount of total impurities present in the lyophilized pharmaceutical composition following 36 months of storage at 20-25° C. does not exceed 0.5%. In one embodiment, the amount of total impurities present in the lyophilized pharmaceutical composition following 36 months of storage at 20-25° C. does not exceed 0.2%.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the lyophilized pharmaceutical composition is free of benzyl alcohol.

In one embodiment, the lyophilized pharmaceutical composition is also free of propylene glycol.

In a preferred embodiment, the lyophilized pharmaceutical composition is free of benzyl alcohol and propylene glycol.

In the sixth aspect, the present disclosure relates to a process of preparing the pharmaceutical composition of phenobarbital or salts thereof that has been reconstituted from a lyophilized pharmaceutical composition of phenobarbital or salts thereof, wherein the pharmaceutical composition has an alcohol content in the range from about 5000 ppm to about 66000 ppm or alternatively from about 5000 ppm to about 70000 ppm, wherein the process comprises dissolving phenobarbital or salts thereof in water to obtain an aqueous solution having a concentration 10-200 mg/ml; lyophilizing the aqueous solution to obtain lyophilized pharmaceutical composition of phenobarbital or salts thereof; and reconstituting the lyophilized pharmaceutical composition to obtain the pharmaceutical composition of phenobarbital or salts thereof.

In one embodiment, the process comprises measuring the alcohol content of an aqueous solution of phenobarbital or salts thereof. In one embodiment, if the alcohol content of the aqueous solution is below 5000 ppm then the process further comprises a step of adding an alcohol in a quantity that is sufficient to achieve the alcohol content of at least about 5000 ppm.

In one embodiment, the process comprises measuring the alcohol content of the lyophilized pharmaceutical composition of phenobarbital or salts thereof. In one embodiment, the process may comprises additional steps of lyophilization to achieve the alcohol content not more than about 66000 ppm or not more than about 70000 ppm (whichever is desired). In one embodiment, if the alcohol content is above 66000 ppm or about 70000 ppm, the process further comprises repeating the lyophilization step until the alcohol content of the lyophilized pharmaceutical composition is not more than about 66000 ppm or not more than about 70000 ppm. It is understood that even a single additional cycle of lyophilization would be sufficient or it has to be repeated n number of times to obtain the desired alcohol level.

In another aspect, the present disclosure relates to a process of preparing the pharmaceutical composition of phenobarbital or salts thereof that has been reconstituted from a lyophilized pharmaceutical composition of phenobarbital or salts thereof, wherein the pharmaceutical composition has an alcohol content in the range from about 5000 ppm to about 66000 ppm or alternatively from about 5000 ppm to about 70000 ppm, wherein the process comprises dissolving phenobarbital or salts thereof in water to obtain an aqueous solution having a concentration 10-200 mg/ml; measuring the alcohol content of aqueous solution; if the alcohol content is below 5000 ppm, adding an alcohol to achieve the alcohol content of at least about 5000 ppm; lyophilizing the aqueous solution to obtain lyophilized pharmaceutical composition; measuring the alcohol content of the lyophilized pharmaceutical composition; if the alcohol content is above 66000 ppm or about 70000 ppm (whichever is desired), repeating the lyophilization step multiple times till the alcohol content of the lyophilized pharmaceutical composition is not more than about 66000 ppm or not more than about 70000 ppm (whichever is desired); and reconstituting the lyophilized pharmaceutical composition to obtain the pharmaceutical composition of phenobarbital or salts thereof.

In one embodiment, the process comprises addition of a pH modifier to the aqueous solution of phenobarbital or salts thereof to achieve a pH in a range of 8-12, preferably in a range of 9-11, more preferably in a range of 9-10.5. In one embodiment, the pH modifier is selected from HCl and/or NaOH. In one embodiment, the pH modifier is aqueous HCl solution.

In one embodiment, the lyophilized pharmaceutical composition is reconstituted with water for injection, an aqueous saline or an aqueous dextrose solution. In one embodiment, the lyophilized pharmaceutical composition is reconstituted with 0.9% aqueous saline.

In one embodiment, the pharmaceutical composition has an alcohol content in the range from about 5000 ppm to about 66000 ppm or alternatively from about 5000 ppm to about 70000 ppm. In one embodiment, the alcohol content is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In one embodiment, the pharmaceutical composition is an aqueous solution for injection of phenobarbital or salts thereof.

In one embodiment, the pharmaceutical composition comprise phenobarbital or salts thereof in a concentration of 10-200 mg/ml. In one embodiment, phenobarbital or salts thereof is present in a concentration of 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml and 200 mg/ml.

In one embodiment, the pharmaceutical composition is stable up to 12 hours of storage at 20-25° C.

In one embodiment, the amount of total impurities in the pharmaceutical composition present following 12 hours of storage at 20-25° C. does not exceed 0.5%. In one embodiment, the amount of total impurities present in the pharmaceutical composition following 12 hours of storage at 20-25° C. does not exceed 0.2%.

In one embodiment, the pharmaceutical composition is stable up to 36 hours of storage at 2-8° C.

In one embodiment, the amount of total impurities present in the pharmaceutical composition following 36 hours of storage at 2-8° C. does not exceed 0.5%. In one embodiment, the amount of total impurities present in the pharmaceutical composition following 36 hours of storage at 2-8° C. does not exceed 0.2%.

Alternatively, the total impurities does not exceed 0.15% following 12 hours of storage at 20-25° C. or 36 hours of storage at 2-8° C., alternatively, the total impurities does not exceed 0.1% following 12 hours of storage at 20-25° C. or 36 hours of storage at 2-8° C., alternatively, the total impurities does not exceed 0.05% following 12 hours of storage at 20-25° C. or 36 hours of storage at 2-8° C.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the osmolality of the pharmaceutical composition is below 500 mOsm/kg.

In one embodiment, the osmolality of the pharmaceutical composition is about 300-400 mOsm/kg.

In one embodiment, the pharmaceutical composition is free of benzyl alcohol.

In one embodiment, the pharmaceutical composition is also free of propylene glycol.

In a preferred embodiment, the pharmaceutical composition is free of benzyl alcohol and propylene glycol.

In the seventh aspect, the present disclosure relates to a lyophilized pharmaceutical composition of phenobarbital or salts thereof having an alcohol content in the range from about 5000 ppm to about 66000 ppm or alternatively from about 5000 ppm to about 70000 ppm, wherein the lyophilized pharmaceutical composition is obtained by a process comprising: dissolving phenobarbital or salts thereof in water to obtain an aqueous solution having a concentration 10-200 mg/ml and lyophilizing the aqueous solution to obtain lyophilized pharmaceutical composition of phenobarbital or salts thereof.

In one embodiment, the process comprises measuring the alcohol content of an aqueous solution of phenobarbital or salts thereof. In one embodiment, if the alcohol content of the aqueous solution is below 5000 ppm then the process further comprises a step of adding an alcohol in a quantity that is sufficient to achieve the alcohol content of at least about 5000 ppm.

In one embodiment, the process comprises measuring the alcohol content of the lyophilized pharmaceutical composition of phenobarbital or salts thereof. In one embodiment, the process may comprises an additional step of lyophilization to achieve the alcohol content not more than about 66000 ppm or not more than 70000 ppm (whichever is desired). In one embodiment, if the alcohol content is above 66000 ppm or about 70000 ppm, the process further comprises repeating the lyophilization step until the alcohol content of the lyophilized pharmaceutical composition is not more than about 66000 ppm or not more than about 70000 ppm (whichever is desired). It is understood that even a single additional cycle of lyophilization would be sufficient or it has to be repeated n number of times to obtain the desired alcohol level.

In one embodiment, the process comprises addition of a pH modifier to the aqueous solution of phenobarbital or salts thereof to achieve a pH in a range of 8-12, preferably in a range of 9-11, more preferably in a range of 9-10.5.

In one embodiment, the pH modifier is selected from HCl and/or NaOH. In one embodiment, the pH modifier is aqueous HCl solution.

In one embodiment, the lyophilized pharmaceutical composition has an alcohol content of at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In one embodiment, the lyophilized pharmaceutical composition is stable up to 36 months of storage at 20-25° C.

In one embodiment, the amount of total impurities present in the lyophilized pharmaceutical composition following 36 months of storage at 20-25° C. does not exceed 0.5%. In one embodiment, the amount of total impurities present in the lyophilized pharmaceutical composition following 36 months of storage at 20-25° C. does not exceed 0.2%.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the lyophilized pharmaceutical composition is free of benzyl alcohol.

In one embodiment, the lyophilized pharmaceutical composition is also free of propylene glycol.

In a preferred embodiment, the lyophilized pharmaceutical composition is free of benzyl alcohol and propylene glycol.

In order to achieve a desired stability, the lyophilized pharmaceutical composition should have an alcohol content in the range from about 5000 ppm to 66000 ppm or alternatively from about 5000 ppm to about 70000 ppm, and thus the process may include the steps as discussed above for measuring the alcohol content, adding alcohol or removing additional alcohol by repeating the lyophilization step and ensuring that the alcohol content is achieved as described above.

In the eight aspect, the present disclosure relates to a pharmaceutical composition of phenobarbital or salts thereof that has been reconstituted from a lyophilized pharmaceutical composition of phenobarbital or salts thereof having an alcohol content in the range from about 5000 ppm to about 66000 ppm or alternatively from about 5000 ppm to about 70000 ppm, wherein the pharmaceutical composition is obtained by a process comprising: dissolving phenobarbital or salts thereof in water to obtain an aqueous solution having a concentration 10-200 mg/ml; lyophilizing the aqueous solution to obtain lyophilized pharmaceutical composition of phenobarbital or salts thereof; and reconstituting the lyophilized pharmaceutical composition to obtain the pharmaceutical composition of phenobarbital or salts thereof.

In one embodiment, the process comprises measuring the alcohol content of an aqueous solution of phenobarbital or salts thereof. In one embodiment, if the alcohol content of the aqueous solution is below 5000 ppm then the process further comprises a step of adding an alcohol in a quantity that is sufficient to achieve the alcohol content of at least about 5000 ppm. In one embodiment, the process comprises measuring the alcohol content of the lyophilized pharmaceutical composition of phenobarbital or salts thereof. In one embodiment, the process may comprises an additional step of lyophilization to achieve the alcohol content not more than about 66000 ppm or not more than about 70000 ppm (whichever is desired). In one embodiment, if the alcohol content is above about 66000 ppm or above about 70000 ppm, the process further comprises repeating the lyophilization step until the alcohol content of the lyophilized pharmaceutical composition is not more than about 66000 ppm or not more than about 70000 ppm (whichever is desired). It is understood that even a single additional cycle of lyophilization would be sufficient or it has to be repeated n number of times to obtain the desired alcohol level.

In one embodiment, the process comprises addition of a pH modifier to the aqueous solution of phenobarbital or salts thereof to achieve a pH in a range of 8-12, preferably in a range of 9-11, more preferably in a range of 9-10.5.

In one embodiment, the pH modifier is selected from HCl and/or NaOH. In one embodiment, the pH modifier is aqueous HCl solution.

In one embodiment, the lyophilized pharmaceutical composition is reconstituted with water for injection, an aqueous saline or an aqueous dextrose solution. In one embodiment, the lyophilized pharmaceutical composition is reconstituted with 0.9% aqueous saline.

In one embodiment, the pharmaceutical composition has an alcohol content in the range from about 5000 ppm to about 66000 ppm or alternatively from about 5000 ppm to about 70000 ppm. In one embodiment, the alcohol content is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In one embodiment, the pharmaceutical composition is an aqueous solution for injection of phenobarbital or salts thereof.

In one embodiment, the pharmaceutical composition comprise phenobarbital or salts thereof in a concentration of 10-200 mg/ml. In one embodiment, phenobarbital or salts thereof is present in a concentration of 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml and 200 mg/ml.

In one embodiment, the pharmaceutical composition is stable up to 12 hours of storage at 20-25° C.

In one embodiment, the amount of total impurities present in the pharmaceutical composition following 12 hours of storage at 20-25° C. does not exceed 0.5%. In one embodiment, the amount of total impurities present in the pharmaceutical composition following 12 hours of storage at 20-25° C. does not exceed 0.2%.

In one embodiment, the pharmaceutical composition is stable up to 36 hours of storage at 2-8° C.

In one embodiment, the amount of total impurities present in the pharmaceutical composition following 36 hours of storage at 2-8° C. does not exceed 0.5%. In one embodiment, the amount of total impurities present in the pharmaceutical composition following 36 hours of storage at 2-8° C. does not exceed 0.2%.

Alternatively, the total impurities does not exceed 0.15% following 12 hours of storage at 20-25° C. or 36 hours of storage at 2-8° C., alternatively, the total impurities does not exceed 0.1% following 12 hours of storage at 20-25° C. or 36 hours of storage at 2-8° C., alternatively, the total impurities does not exceed 0.05% following 12 hours of storage at 20-25° C. or 36 hours of storage at 2-8° C.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the osmolality of the pharmaceutical composition is below 500 mOsm/kg.

In one embodiment, the osmolality of the pharmaceutical composition is about 300-400 mOsm/kg.

In one embodiment, the pharmaceutical composition is free of benzyl alcohol.

In one embodiment, the pharmaceutical composition is also free of propylene glycol.

In a preferred embodiment, the pharmaceutical composition is free of benzyl alcohol and propylene glycol.

In order to achieve a desired stability, the pharmaceutical composition should have an alcohol content in the range from about 5000 ppm to 66000 ppm or alternatively from about 5000 ppm to about 70000 ppm, and thus the process may include the steps as discussed above for measuring the alcohol content, adding alcohol or removing additional alcohol by repeating the lyophilization step and ensuring that the alcohol content is achieved as described above.

In the ninth aspect, the present disclosure relates to a lyophilized pharmaceutical composition comprising phenobarbital or salts thereof and an alcohol.

In one embodiment, the alcohol is present in an amount sufficient to inhibit phenobarbital degradation, such that the amount of total impurities present in the lyophilized pharmaceutical composition following 36 months of storage at 20-25° C. does not exceed 0.5%. In one embodiment, the amount of total impurities present in the lyophilized pharmaceutical composition following 36 months of storage at 20-25° C. does not exceed 0.2%.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the amount of alcohol sufficient to inhibit phenobarbital degradation is in the range from about 5000 ppm to about 66000 ppm or alternatively from about 5000 ppm to about 70000 ppm. In one embodiment, the alcohol content is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In one embodiment, the lyophilized pharmaceutical composition is stable up to 36 months of storage at 20-25° C.

In one embodiment, the lyophilized pharmaceutical composition is free of benzyl alcohol.

In one embodiment, the lyophilized pharmaceutical composition is also free of propylene glycol.

In a preferred embodiment, the lyophilized pharmaceutical composition is free of benzyl alcohol and propylene glycol.

In one embodiment, the present disclosure relates to a lyophilized pharmaceutical composition comprising phenobarbital sodium and ethanol, wherein ethanol is present in an amount sufficient to inhibit degradation of phenobarbital sodium, such that the amount of total impurities present in the lyophilized pharmaceutical composition following 36 months of storage at 20-25° C. does not exceed 0.2%;
wherein the amount of ethanol sufficient to inhibit degradation of phenobarbital sodium is in the range from about 12000 ppm to about 25000 ppm;
and wherein the pharmaceutical composition is free of benzyl alcohol and propylene glycol.

In the tenth aspect, the present disclosure relates to an aqueous solution for injection comprising phenobarbital or salts thereof and an alcohol.

In one embodiment, the aqueous solution for injection is reconstituted from the lyophilized pharmaceutical composition of phenobarbital or salts thereof.

In one embodiment, the lyophilized pharmaceutical composition is reconstituted with water for injection, an aqueous saline or an aqueous dextrose solution.

In one embodiment, the alcohol is present in an amount sufficient to inhibit phenobarbital degradation, such that the amount of total impurities present in the aqueous solution following 12 hours of storage at 20-25° C. does not exceed 0.5%. In one embodiment, the amount of total impurities present in the aqueous solution following 12 hours of storage at 20-25° C. does not exceed 0.2%.

In one embodiment, the alcohol is present in an amount sufficient to inhibit phenobarbital degradation, such that the amount of total impurities present in the aqueous solution following 36 hours of storage at 2-8° C. does not exceed 0.5%. In one embodiment, the amount of total impurities present in the aqueous solution following 36 hours of storage at 2-8° C. does not exceed 0.2%.

Alternatively, the total impurities does not exceed 0.15% following 12 hours of storage at 20-25° C. or 36 hours of storage at 2-8° C., alternatively, the total impurities does not exceed 0.1% following 12 hours of storage at 20-25° C. or 36 hours of storage at 2-8° C., alternatively, the total impurities does not exceed 0.05% following 12 hours of storage at 20-25° C. or 36 hours of storage at 2-8° C.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the amount of alcohol sufficient to inhibit phenobarbital degradation is in the range from about 5000 ppm to about 66000 ppm or alternatively from about 5000 ppm to about 70000 ppm. In one embodiment, the alcohol content is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm.

In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In one embodiment, the aqueous solution comprise phenobarbital or salts thereof in a concentration of 10-200 mg/ml. In one embodiment, phenobarbital or salts thereof is present in a concentration of 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml and 200 mg/ml.

In one embodiment, the aqueous solution is stable up to 12 hours of storage at 20-25° C.

In one embodiment, the aqueous solution is stable up to 36 hours of storage at 2-8° C.

In one embodiment, the osmolality of the aqueous solution is below 500 mOsm/kg. In one embodiment, the osmolality of the aqueous solution is about 300-400 mOsm/kg.

In one embodiment, the aqueous solution is free of benzyl alcohol.

In one embodiment, the aqueous solution is also free of propylene glycol.

In a preferred embodiment, the aqueous solution is free of benzyl alcohol and propylene glycol.

In one embodiment, the present disclosure relates to an aqueous solution for injection comprising phenobarbital sodium and ethanol, wherein ethanol is present in an amount sufficient to inhibit degradation of phenobarbital sodium, such that the amount of total impurities present in the aqueous solution following 12 hours of storage at 20-25° C. or following 36 hours of storage at 2-8° C. does not exceed 0.2%;
wherein the amount of ethanol sufficient to inhibit degradation of phenobarbital sodium is in the range from about 12000 ppm to about 25000 ppm;
wherein phenobarbital sodium is present in a concentration from 10-200 mg/ml;
wherein the aqueous solution is reconstituted from the lyophilized pharmaceutical composition of phenobarbital sodium;
and wherein the aqueous solution is free of benzyl alcohol and propylene glycol.

In the eleventh aspect, the present disclosure relates to a process for the preparation of the pharmaceutical composition of phenobarbital or salts thereof, wherein the process comprises dissolving phenobarbital or salt thereof in water to obtain an aqueous solution having a concentration 10-200 mg/ml and lyophilizing the aqueous solution to obtain lyophilized pharmaceutical composition, wherein phenobarbital or salts thereof has an alcohol content in the range from about 5000 ppm to about 66000 ppm or alternatively from about 5000 ppm to about 70000 ppm.

In one embodiment, the process comprises phenobarbital or salts thereof having an alcohol content in the range from about 5000 ppm to about 66000 ppm or alternatively from about 5000 ppm to about 70000 ppm. In one embodiment, the active pharmaceutical ingredient of phenobarbital or salts thereof for the purpose of this aspect is having an alcohol content in the range from about 5000 ppm to about 66000 ppm or alternatively from about 5000 ppm to about 70000 ppm.

In one embodiment, the process further comprises reconstitution of the lyophilized pharmaceutical composition. In one embodiment, the lyophilized pharmaceutical composition is reconstituted with water for injection, an aqueous saline or an aqueous dextrose solution. In one embodiment, the lyophilized pharmaceutical composition is reconstituted with 0.9% aqueous saline.

In one embodiment, phenobarbital or salts thereof has an alcohol content of at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In one embodiment, the pharmaceutical composition is free of benzyl alcohol.

In one embodiment, the pharmaceutical composition is also free of propylene glycol.

In a preferred embodiment, the pharmaceutical composition is free of benzyl alcohol and propylene glycol.

In one embodiment, the pharmaceutical composition is lyophilized pharmaceutical composition of phenobarbital or salts thereof.

In one embodiment, the lyophilized pharmaceutical composition is stable up to 36 months of storage at 20-25° C.

In one embodiment, the amount of total impurities present in the lyophilized pharmaceutical composition following 36 months of storage at 20-25° C. does not exceed 0.5%. In one embodiment, the amount of total impurities present in the lyophilized pharmaceutical composition following 36 months of storage at 20-25° C. does not exceed 0.2%.

In one embodiment, the pharmaceutical composition is an aqueous solution for injection of phenobarbital or salts thereof.

In one embodiment, the aqueous solution comprise phenobarbital or salts thereof in a concentration of 10-200 mg/ml. In one embodiment, phenobarbital or salts thereof is present in a concentration of 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml and 200 mg/ml.

In one embodiment, the aqueous solution is stable up to 12 hours of storage at 20-25° C.

In one embodiment, the amount of total impurities present in the aqueous solution following 12 hours of storage at 20-25° C. does not exceed 0.5%. In one embodiment, the amount of total impurities present in the aqueous solution following 12 hours of storage at 20-25° C. does not exceed 0.2%.

In one embodiment, the aqueous solution is stable up to 36 hours of storage at 2-8° C.

In one embodiment, the amount of total impurities present in the aqueous solution following 36 hours of storage at 2-8° C. does not exceed 0.5%. In one embodiment, the amount of total impurities present in the aqueous solution following 36 hours of storage at 2-8° C. does not exceed 0.2%. Alternatively, the total impurities does not exceed 0.15% following 12 hours of storage at 20-25° C. or 36 hours of storage at 2-8° C., alternatively, the total impurities does not exceed 0.1% following 12 hours of storage at 20-25° C. or 36 hours of storage at 2-8° C., alternatively, the total impurities does not exceed 0.05% following 12 hours of storage at 20-25° C. or 36 hours of storage at 2-8° C.

In one embodiment, the osmolality of the aqueous solution is below 500 mOsm/kg. In one embodiment, the osmolality of the aqueous solution is about 300-400 mOsm/kg.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In another aspect, the present disclosure relates to a lyophilized pharmaceutical composition of phenobarbital or salts thereof, wherein the lyophilized pharmaceutical composition has: (i) an amount of alcohol present following 36 months of storage at 20-25° C. in the range from about 5000 ppm to about 66000 ppm or alternatively from about 5000 ppm to about 70000 ppm; or (ii) an amount of total impurities present following 36 months of storage at 20-25° C. does not exceed 0.2%.

In one embodiment, the lyophilized pharmaceutical composition has an amount of alcohol present following 36 months of storage at 20-25° C. in the range from about 5000 ppm to about 66000 ppm or alternatively from about 5000 ppm to about 70000 ppm. In one embodiment, the alcohol content is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In one embodiment, the lyophilized pharmaceutical composition is stable up to 36 months of storage at 20-25° C.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the lyophilized pharmaceutical composition is free of benzyl alcohol.

In one embodiment, the lyophilized pharmaceutical composition is also free of propylene glycol.

In a preferred embodiment, the lyophilized pharmaceutical composition is free of benzyl alcohol and propylene glycol.

In another aspect, the present disclosure relates to a pharmaceutical composition of phenobarbital or salts thereof that has been reconstituted from a lyophilized pharmaceutical composition of phenobarbital or salts thereof, wherein the pharmaceutical composition has: (i) an amount of alcohol present following 12 hours of storage at 20-25° C. or 36 hours of storage at 2-8° C. in the range from about 5000 ppm to about 66000 ppm or alternatively from about 5000 ppm to about 70000 ppm; or (ii) an amount of total impurities present following 12 hours of storage at 20-25° C. or 36 hours of storage at 2-8° C. does not exceed 0.2%.

In one embodiment, the pharmaceutical composition has an amount of alcohol present following 12 hours of storage at 20-25° C. or 36 hours of storage at 2-8° C. in the range from about 5000 ppm to about 66000 ppm or alternatively from about 5000 ppm to about 70000 ppm. In one embodiment, the alcohol content is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In one embodiment, the lyophilized pharmaceutical composition is reconstituted with water for injection, an aqueous saline or an aqueous dextrose solution. In one embodiment, the lyophilized pharmaceutical composition is reconstituted with 0.9% aqueous saline.

In one embodiment, the pharmaceutical composition is an aqueous solution for injection of phenobarbital or salts thereof.

In one embodiment, the pharmaceutical composition comprise phenobarbital or salts thereof in a concentration of 10-200 mg/ml. In one embodiment, phenobarbital or salts thereof is present in a concentration of 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml and 200 mg/ml.

In one embodiment, the pharmaceutical composition is stable up to 12 hours of storage at 20-25° C.

In one embodiment, the pharmaceutical composition is stable up to 36 hours of storage at 2-8° C.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the osmolality of said pharmaceutical composition is below 500 mOsm/kg.

In one embodiment, the osmolality of said aqueous solution is about 300-400 mOsm/kg.

In one embodiment, the pharmaceutical composition is free of benzyl alcohol.

In one embodiment, the pharmaceutical composition is also free of propylene glycol.

In a preferred embodiment, the pharmaceutical composition is free of benzyl alcohol and propylene glycol.

In another aspect, the present disclosure relates to a lyophilized pharmaceutical composition of phenobarbital sodium, wherein the composition has an ethanol content in the range from about 12000 ppm to about 25000 ppm; wherein the composition is stable up to 36 months of storage at 20-25° C. such that the amount of total impurities present following 36 months of storage at 20-25° C. does not exceed 0.2%; and wherein the composition is free of benzyl alcohol and propylene glycol.

In another aspect, the present disclosure relates to an aqueous solution for injection of phenobarbital sodium, wherein the aqueous solution has an ethanol content in the range from about 12000 ppm to about 25000 ppm; wherein the aqueous solution is stable up to 12 hours of storage at 20-25° C. or 36 hours of storage at 2-8° C. such that the amount of total impurities present following 12 hours of storage at 20-25° C. or following 36 hours of storage at 2-8° C. does not exceed 0.2%; alternatively, the total impurities does not exceed 0.15% following 12 hours of storage at 20-25° C. or 36 hours of storage at 2-8° C.; alternatively, the total impurities does not exceed 0.1% following 12 hours of storage at 20-25° C. or 36 hours of storage at 2-8° C.; alternatively, the total impurities does not exceed 0.05% following 12 hours of storage at 20-25° C. or 36 hours of storage at 2-8° C.; wherein the aqueous solution is reconstituted from the lyophilized pharmaceutical composition of phenobarbital sodium; and wherein the aqueous solution is free of benzyl alcohol and propylene glycol.

In another aspect, the present disclosure relates to a method of preventing degradation of phenobarbital or salts thereof, wherein the method comprises dissolving phenobarbital or salts thereof in water to obtain an aqueous solution having a concentration 10-200 mg/ml; and lyophilizing the aqueous solution to obtain lyophilized pharmaceutical composition of phenobarbital or salts thereof, wherein the method comprises presence of alcohol, in an amount sufficient to inhibit the degradation of phenobarbital or salts thereof.

In one embodiment, the present disclosure relates to reconstituting the lyophilized pharmaceutical composition to obtain the aqueous solution for injection of phenobarbital or salts thereof.

In one embodiment, wherein the amount of alcohol sufficient to inhibit degradation of phenobarbital or salts thereof is in the range from about 5000 ppm to about 66000 ppm or alternatively from about 5000 ppm to about 70000 ppm. In one embodiment, the amount of alcohol is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

In yet another aspect, the present invention relates to phenobarbital or salts thereof having an alcohol in an amount sufficient to inhibit degradation of phenobarbital sodium.

In one embodiment, the amount of alcohol sufficient to inhibit degradation of phenobarbital or salts thereof is such that when said phenobarbital or salts thereof having an alcohol is stored for 36 months at 20-25° C., the amount of total impurities does not exceed 0.5%, preferably does not exceed 0.2%.

In an alternate embodiment, the amount of alcohol sufficient to inhibit degradation of phenobarbital or salts thereof is such that when said phenobarbital or salts thereof is dissolved in an aqueous media and is stored for 12 hours at 20-25° C. or 36 hours at 2-8° C., the amount of total impurities does not exceed 0.5%, preferably does not exceed 0.2%. In one embodiment, the amount of alcohol sufficient to inhibit degradation of phenobarbital or salts thereof is from about 5000 ppm to about 70000 ppm.

In one embodiment, the phenobarbital or salts thereof having an alcohol content in a range from about 5000 ppm to about 70000 ppm, is stable up to 36 months of storage at 20-25° C. such that the amount of total impurities present following 36 months of storage at 20-25° C. does not exceed 0.5%, preferably does not exceed 0.2%.

In another embodiment, the phenobarbital or salts thereof having an alcohol content in a range from about 5000 ppm to about 70000 ppm, when dissolved in an aqueous media, the resulting aqueous solution of phenobarbital or salts thereof remains stable up to 12 hours of storage at 20-25° C. or 36 hours of storage at 2-8° C. such that the amount of total impurities present following 12 hours of storage at 20-25° C. or following 36 hours of storage at 2-8° C. does not exceed 0.5%, preferably does not exceed 0.2%.

In one embodiment, the amount of alcohol is at least about 5000 ppm, about 10000 ppm, about 15000 ppm, about 20000 ppm, about 25000 ppm, about 30000 ppm, about 35000 ppm, about 40000 ppm, about 45000 ppm, about 50000 ppm, about 55000 ppm, about 60000 ppm, about 65000 ppm, about 66000 ppm, or about 70000 ppm. In another embodiment, the alcohol content is in the range from about 12000 ppm to about 70000 ppm, about 12000 ppm to about 66000 ppm, about 12000 ppm to about 65000 ppm, about 12000 ppm to about 60000 ppm, about 12000 ppm to about 55000 ppm, about 12000 ppm to about 50000 ppm, about 12000 ppm to about 45000 ppm, about 12000 ppm to about 40000 ppm, about 12000 ppm to about 35000 ppm, about 12000 ppm to about 30000 ppm, about 12000 ppm to about 25000 ppm, about 12000 ppm to about 20000 ppm or about 12000 ppm to about 15000 ppm. In another embodiment, the alcohol content is in the range from about 15000 ppm to about 70000 ppm, about 15000 ppm to about 66000 ppm, about 25000 ppm to about 66000 ppm, about 35000 to about 66000 ppm, about 45000 ppm to about 66000 ppm, about 55000 ppm to about 66000 ppm, about 35000 ppm to about 55000 ppm or about 30000 ppm to about 50000 ppm.

In another embodiment, the alcohol content is in the range from about 12000 ppm to about 25000 ppm. In one embodiment, the alcohol content is in the range from about 35000 to about 66000 ppm.

In one embodiment, the alcohol is a C₁-C₃ alcohol.

In one embodiment, the alcohol is ethanol.

In one embodiment, phenobarbital or salts thereof is phenobarbital base or phenobarbital sodium, preferably phenobarbital sodium.

Through lyophilization, the present inventors have found that the alcohol content of phenobarbital sodium may be reduced to less than 50000 ppm. The present inventors have found that in some embodiments, the alcohol content may be reduced to a level of about 5000 ppm. In other embodiments, the alcohol content may be reduced to a level of about 12000 ppm to about 25000 ppm. The present inventors have found that the low temperature vacuum drying conditions afforded by lyophilization provide a safe and effective mechanism for converting the tested material to a solid state. Lyophilization, also known as freeze-drying, may consist of three separate, unique, and interdependent process; freezing, primary drying (sublimation) and secondary drying (desorption). In certain preferred embodiments, the step of freezing the aqueous solution may occur at a temperature range of −20° C. to −50° C. In certain preferred embodiments, the step of freezing the aqueous solution may occur at a temperature range of about −45° C. In certain preferred embodiments, the step drying the aqueous solution may occur at primary and/or secondary temperature ranges of −35 to 25° C. In certain preferred embodiments, the step drying the aqueous solution may occur at primary and/or secondary temperature ranges of −45 to 45° C. In certain preferred embodiments, the step drying the aqueous solution may occur at primary and/or secondary temperature above the collapse temperature (Tc) of phenobarbital or a salt thereof. In certain preferred embodiments, the step drying the aqueous solution may occur at primary temperature above the collapse temperature (Tc) of phenobarbital or a salt thereof (i.e., above −24.4±0.5° C.). In certain preferred embodiments, the step drying the aqueous solution may occur at a primary temperature in the range of from about −40° C. to about 20° C. In certain preferred embodiments, the step drying the aqueous solution may occur at a secondary temperature in the range of from about 20° C. to about 45° C. However, it should be understood that these temperatures are exemplary only, and temperatures may be chosen so as to optimize the lyophilized powder. Where secondary temperature ranges are used, the step of drying may be split into two separate steps, each occurring at a different temperature and a different time. In certain embodiments, there may also be performed a step of annealing the aqueous solution, which may occur at a temperature range of −15° C. to −25° C. In certain embodiments, the lyophilization process does not include annealing. In certain embodiments, a desired level of residual water content after lyophilization may be less than 3% w/w.

Lyophilized product is generally obtained by following process:
Preparation of solution of active with or without excipeints. Excipeints are added to provide pharmaceutically elegant, mechanically strong cake which does not interfere with reconstitution or improve reconstitution;
Filteration;
Filling in lyophilization containers, typically glass vials;
Lyophilization—using an optimal lyophilzation process consiting of single or multi-step freezing, annealing, primary drying (sublimation), and secondary drying (desorption);
The cake so formed should be rapidly reconstituted without residual particles.

Primary drying is typically the longest step in a lyophilization process. For many years, cycle and formulation optimization was performed to assure that the product temperature during primary drying would never exceed the collapse temperature. The collapse temperature is the product temperature during freeze-drying above which product cake begins to lose its original structure. It was reported in literature that, above the collapse temperature, product could experience slow sporadic bubbling, swelling, foaming, cavitation, fenestration, gross collapse, retraction and beading that may have consequences on the appearance of the product (MacKenzie, “Collapse during freeze-drying-Qualitative and quantitative aspects” In Freeze-Drying and Advanced Food Technology; Goldblith, S. A., Rey. L, Rothmayr, W. W., Eds.; Academic Press, New York, 1974, 277-307). As a result, it is thought that collapse results in poor product stability, long drying times (due to pore's collapse), uneven drying and loss of texture (R. Bellows, et al. “Freeze-drying of aqueous solutions: maximum allowable operating temperature,” Cryobiology, 9, 559-561 (1972).

The present inventors discovered that the primary drying may be carried out above the collapse temperature while still retaining product stability, and other important product attributes. Thus, the present invention provides, improved lyophilization methods with significantly shortened primary drying step, providing significant cost benefits.

In the twelfth aspect, the present disclosure relates to a method for the preparation of the lyophilized pharmaceutical composition of phenobarbital or a salt thereof, wherein the method includes (a) dissolving phenobarbital or a salt thereof in water to obtain a pre-lyophilization solution; and (b) lyophilizing the pre-lyophilized solution to obtain a lyophilized pharmaceutical composition of phenobarbital or a salt thereof.

In one embodiment, the method includes dissolving phenobarbital sodium in water to obtain a pre-lyophilization solution having a concentration of phenobarbital or a salt thereof (e.g., phenobarbital sodium) of about 10 mg/ml to about 60 mg/ml, including about 15 mg/ml to about 55 mg/ml, about 20 mg/ml to about 50 mg/ml, about 25 mg/ml to about 45 mg/ml, or about 30 mg/ml to about 40 mg/ml.

In one embodiment, the step of lyophilizing the pre-lyophilized solution comprises: (i) freezing a pre-lyophilized solution to a freezing temperature to form a frozen solution; (ii) holding the frozen solution at or below the freezing temperature, for a first time period; (iii) evacuating and ramping the frozen solution to a primary drying temperature to form a dried solution, wherein the primary drying temperature is higher than the collapse temperature of phenobarbital or a salt thereof, (iv) holding the first dried material for a second time period, under a first pressure; (v) ramping the dried solution to a secondary drying temperature; and (vi) holding for a third time period, under a second pressure to form a lyophilized composition of phenobarbital or salts thereof.

In one embodiment, the method includes freezing the pre-lyophilized solution to a freezing temperature ranging from about 5° C. to about −50° C., including about 5° C. to about −45° C., about 0° C. to about −40° C., about −5° C. to about −35° C., or about −10° C. to about −30° C. In one embodiment, the method includes freezing the pre-lyophilized solution to a freezing temperature of about −45° C.

In one embodiment, the method includes holding the frozen solution at or below the freezing temperature of below −50° C., including below −45° C., below −40° C., below −35° C., or below −30° C.

In one embodiment, the method includes holding the frozen solution at or below the freezing temperature for a period of less than about 500 min, including less than about 450 min, less than about 400 min, less than about 350 min or less than about 300 min. In one embodiment, the method includes holding the frozen solution at or below the freezing temperature for a period of about 500 min, about 450 min, about 400 min, about 350 min, about 300 min or about 200 min. In one embodiment, the method includes holding the frozen solution at or below the freezing temperature for a period of about 100 min to about 500 min, including about 200 min to about 400 min, about 300 min to about 400 min, or about 300 min to about 350 min.

In one embodiment, the method includes holding the frozen solution at or below −45° C. for about 200 min to about 500 min. In another embodiment, the method includes holding the frozen solution at or below −45° C. for about 300 min.

In one embodiment, the method includes evacuating and ramping the frozen solution to a primary drying temperature to form a dried solution, wherein the primary drying temperature is higher than the collapse temperature of phenobarbital sodium. In one embodiment, the primary drying temperature is greater than about −25° C., including greater than about −20° C., greater than about −10° C., greater than about 0° C., greater than about 10° C. or greater than about 20° C. In one embodiment, the primary drying temperature ranges from about −20° C. to about 30° C., about −10° C. to about 25° C., about 0° C. to about 20° C., or about 10° C. to about 20° C. In one embodiment, the primary drying temperature is about 0° C., about 5° C., about 10° C., about 15° C., about 20° C., or about 25° C. In one embodiment, the primary drying temperature is about 20° C.

Following the primary drying, the first dried material is held for a second time period, under a first pressure. In one embodiment, the method includes holding the first dried material for a period of less than about 1000 min, including less than about 950 min, less than about 900 min or less than about 850 min. In one embodiment, the method includes holding the first dried material for a period of about 1000 min, including about 950 min, about 900 min, about 850 min, about 800 min, about 750 min, about 700 min, about 650 min, about 600 min, about 550 min, about 500 min, about 450 min, about 400 min, about 350 min, or about 300 min. In one embodiment, the method includes the first dried material for a period of about 100 min to about 1000 min, including about 200 min to about 1000 min, about 300 min to about 950 min, about 400 min to about 900 min, or about 500 min to about 850 min.

In one embodiment, the method includes holding the first dried material under a first pressure of less than about 120 mTorr, less than about 100 mTorr, less than about 80 mTorr, or less than about 60 mTorr. In one embodiment, the method includes holding the first dried material under a first pressure of about 20 to about 200 mTorr, including about 30 to about 150 mTorr, about 35 to about 120 mTorr, or about 40 to about 100 mTorr. In one embodiment, the method includes holding the first dried material under a first pressure of about 35 to about 120 mTorr.

In one embodiment, the method includes holding the first dried for less than about 900 min, under a pressure of less than about 60 mTorr.

In one embodiment, the method includes ramping the dried solution to a secondary drying temperature. In one embodiment, the secondary drying temperature is greater than about −20° C., including greater than about 10° C., greater than about 20° C., greater than about 30° C. or greater than about 40° C. In one embodiment, the secondary drying temperature ranges from about 5° C. to about 50° C., about 10° C. to about 45° C., about 20° C. to about 40° C., or about 30° C. to about 35° C. In one embodiment, the secondary drying temperature is about 10° C., about 15° C., about 20° C., about 25° C., about 30° C., about 35° C., about 40° C., or about 45° C. In one embodiment, the secondary drying temperature is about 45° C.

Following the secondary drying, the second dried material is held for a third time period, under a second pressure. In one embodiment, the method includes holding the second dried material for a period of less than about 500 min, including less than about 450 min, less than about 400 min or less than about 350 min. In one embodiment, the method includes holding the second dried material for a period of about 100 min, including about 150 min, about 200 min, about 250 min, about 300 min, about 310 min, about 320 min, about 330 min, about 340 min, about 350 min, about 360 min, about 375 min, about 380 min, about 390 min, or about 400 min. In one embodiment, the method includes the second dried material for a period of about 100 min to about 500 min, including about 250 min to about 450 min, about 275 min to about 450 min, or about 300 min to about 350.

In one embodiment, the method includes holding the second dried material under a second pressure of about 10 to about 100 mTorr, including about 20 to about 80 mTorr, about 35 to about 60 mTorr, or about 40 to about 50 mTorr. In one embodiment, the method includes holding the second dried material under a first pressure of about 35 to about 60 mTorr. In one embodiment, holding for about 200 min to about 400 min, under a pressure of about 35 to about 60 mTorr; to form a lyophilized composition. In one embodiment, the method includes holding the second dried material for about 330 min, under a pressure of about 50 mTorr.

In the thirteenth aspect, the present disclosure relates to a method for preparation of the lyophilized pharmaceutical composition of phenobarbital sodium comprising: (a) dissolving phenobarbital sodium in water to obtain a pre-lyophilization solution having a concentration between 10 mg/ml to 60 mg/ml of phenobarbital sodium; and (b) lyophilizing the pre-lyophilized solution to obtain a lyophilized pharmaceutical composition.

In one embodiment, step (b) of the method includes (i) freezing a pre-lyophilized solution of step (a) to a temperature from about 5° C. to about −45° C. to form a frozen solution; (ii) holding the frozen solution at or below −45° C., for about 200 min to about 500 min; (iii) evacuating and ramping the frozen solution to a primary drying temperature to form a dried solution, wherein the primary drying temperature is higher than the collapse temperature of phenobarbital sodium; (iv) holding for less than about 1000 min, under a pressure of about 35 to about 120 mTorr; (v) ramping the dried solution to a secondary drying temperature from about 20° C. to about 45° C.; and (vi) holding for about 200 min to about 400 min, under a pressure of about 35 to about 60 mTorr; to form a lyophilized composition of phenobarbital sodium.

In another embodiment, step (b) of the method includes (i) freezing a pre-lyophilization solution of step (a) to a temperature of about −45° C. to form a frozen solution; (ii) holding the frozen solution at or below −45° C., for about 300 min; (iii) evacuating and ramping the frozen solution to a primary drying temperature of about 20° C. to form a dried solution; (iv) holding for less than about 900 min, under a pressure of less than about 60 mTorr; (v) ramping the dried solution to a secondary drying temperature of about 45° C.; and (vi) holding for about 330 min, under a pressure of about 50 mTorr; to form a lyophilized pharmaceutical composition of phenobarbital sodium.

In one embodiment, the lyophilized pharmaceutical composition obtained by the method has a percent porosity in the range from about 20% to about 50%.

In one embodiment, the lyophilized pharmaceutical composition obtained by the method has a percent porosity in the range from about 22% to about 48%.

In one embodiment, the lyophilized pharmaceutical composition obtained by the method has a percent porosity in the range from about 25% to about 45%.

In one embodiment, the lyophilized pharmaceutical composition obtained by the method further comprises an ethanol content in the range from about 12000 ppm to about 25000 ppm, about 12000 ppm to about 20000 ppm or about 12000 ppm to about 15000 ppm.

In one embodiment, the lyophilized pharmaceutical composition obtained by the method comprises total impurities in an amount of less than 0.5%, preferably less than 0.2%, following 36 months of storage at about 20° C. to about 25° C. Alternatively, the total impurities does not exceed 0.15% following 12 hours of storage at 20-25° C. or 36 hours of storage at 2-8° C., alternatively, the total impurities does not exceed 0.1% following 12 hours of storage at 20-25° C. or 36 hours of storage at 2-8° C., alternatively, the total impurities does not exceed 0.05% following 12 hours of storage at 20-25° C. or 36 hours of storage at 2-8° C.

In one embodiment, the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the lyophilized pharmaceutical composition obtained by the method is free of benzyl alcohol and propylene glycol.

In the fourteenth aspect, the present disclosure relates to a lyophilized pharmaceutical composition of phenobarbital sodium, prepared by a method according to the method of twelfth aspect or thirteenth aspect.

In one embodiment, the lyophilized pharmaceutical composition has a percent porosity in the range from about 20% to about 50%, including a percent porosity in the range from about 20% to about 45%, from about 22% to about 45%, from about 25% to about 45%, from about 30% to about 45%, or from about 35% to about 45%. In one embodiment, the lyophilized phenobarbital sodium has a percent porosity in the range from about 20% to about 50%. In one embodiment, the lyophilized phenobarbital sodium has a percent porosity in the range from about 22% to about 50%. In one embodiment, the lyophilized phenobarbital sodium has a percent porosity in the range from about 25% to about 50%. In one embodiment, the lyophilized phenobarbital sodium has a percent porosity in the range from about 30% to about 50%. In one embodiment, the lyophilized phenobarbital sodium has a percent porosity in the range from about 22% to about 48%. In one embodiment, the lyophilized phenobarbital sodium has a percent porosity in the range from about 25% to about 45%.

In one embodiment, the lyophilized pharmaceutical composition further comprises an ethanol content in the range from about 12000 ppm to about 25000 ppm, about 12000 ppm to about 20000 ppm or about 12000 ppm to about 15000 ppm.

In one embodiment, the lyophilized pharmaceutical composition comprises total impurities in an amount of less than 0.5%, preferably less than 0.2% following 36 months of storage at about 20° C. to about 25° C. Alternatively, the total impurities does not exceed 0.15% following 12 hours of storage at 20-25° C. or 36 hours of storage at 2-8° C., alternatively, the total impurities does not exceed 0.1% following 12 hours of storage at 20-25° C. or 36 hours of storage at 2-8° C., alternatively, the total impurities does not exceed 0.05% following 12 hours of storage at 20-25° C. or 36 hours of storage at 2-8° C.

In one embodiment, the impurities are selected from the group consisting of 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

In one embodiment, the lyophilized pharmaceutical composition is free of benzyl alcohol and propylene glycol.

In one embodiment, the lyophilized pharmaceutical composition is reconstituted with water for injection, an aqueous saline or an aqueous dextrose solution.

In one embodiment, the lyophilized pharmaceutical composition includes phenobarbital sodium in a concentration of 10-200 mg/ml.

In one embodiment, the lyophilized pharmaceutical composition has an osmolality below 500 mOsm/kg. In one embodiment, the osmolality of said pharmaceutical composition is about 300-400 mOsm/kg.

In another aspect, the present disclosure relates to an injectable formulation comprising phenobarbital sodium, reconstituted from the lyophilized pharmaceutical composition of the fourteenth aspect in a sterile carrier suitable for intravenous administration.

In the fifteenth aspect, the present disclosure relates to lyophilized pharmaceutical composition of phenobarbital sodium having one or more of the following attributes:
(a) a percent porosity in the range from about 20% to about 50%;
(b) an ethanol content in the range from about 12000 ppm to about 25000 ppm;
(c) total impurities in an amount of less than 0.2% following 36 months of storage at about 20° C. to about 25° C.;
(d) free of benzyl alcohol and propylene glycol; and
(e) lyophilized phenobarbital sodium in an amount from 10 mg/vial to 100 mg/vial.

In one embodiment, the lyophilized pharmaceutical composition comprises lyophilized phenobarbital sodium in an amount of 10 mg/vial. In another embodiment, the lyophilized pharmaceutical composition comprises lyophilized phenobarbital sodium in an amount of 100 mg/vial.

In sixteenth aspect, the present disclosure relates to pharmaceutical composition of phenobarbital sodium made by a method comprising reconstituting a lyophilized pharmaceutical composition of the fourteenth aspect or the fifteenth aspect.

In another aspect, the lyophilized pharmaceutical composition of phenobarbital sodium according to the present invention provides improved contact area available to the vehicle and increases both the rate and extent of dissolution.

In another aspect, the present invention provides a lyophilized pharmaceutical composition of phenobarbital sodium that dissolves relatively quickly within 10-60 seconds, preferably within 20-40 seconds, more preferably within 10-30 seconds in a modest amount of vehicle such as 0.9% of aqueous saline, dextrose or water for injection.

In the seventeenth aspect, the present disclosure relates to a method for the treatment of neonatal seizure in newborn infants of 2 weeks of age or younger in need thereof, comprising administering the pharmaceutical composition of the sixteenth aspect.

In one embodiment, the method comprises reconstituting the lyophilized pharmaceutical composition immediately prior to the administration. In one embodiment, the lyophilized pharmaceutical composition is reconstituted with water for injection, an aqueous saline or an aqueous dextrose solution.

In one aspect, the present technology provides methods of lyophilizing a liquid formulation including a primary drying step executed at a product temperature at or above the collapse temperature. In one embodiment, the inventive methods include a primary drying step executed without avoiding collapse (e.g., micro-collapse, visually detectable, or macro-collapse) in the lyophilized products.

In some embodiments, the liquid formulation contains a pharmaceutical substance (e.g., phenobarbital or salts therein) at a concentration of at least about 10 mg/ml (e.g., at least about 10 mg/ml, at least about 50 mg/ml, at least about 100 mg/ml, at least about 150 mg/ml, at least about 200 mg/ml, at least about 250 mg/ml, at least about 300 mg/ml, or at least about 400 mg/ml). In other embodiments, the liquid formulation containing a pharmaceutical substance are used at concentrations of about 10 mg/ml to about 60 mg/ml, about 15 mg/ml to about 55 mg/ml, about 20 mg/ml to about 50 mg/ml, about 25 mg/ml to about 45 mg/ml, or about 30 mg/ml to about 40 mg/ml.

In another aspect, the present invention provides a lyophilization process that provides higher surface area and porosity of the cake.

In one embodiment, the percent porosity of the lyophilized cake maybe greater than about 20%, greater than about 22%, greater than about 23%, greater than about 24%, greater than about 25%, greater than about 26%, greater than about 27%, greater than about 28%, greater than about 29%, greater than about 30%, greater than about 35%, or greater than about 40%. In one embodiment, the percent porosity of the lyophilized cake may range from about 20% to about 50%, including a percent porosity in the range from about 22% to about 48%, including a percent porosity in the range from about 20% to about 45%, from about 25% to about 45%, from about 30% to about 45%, or from about 35% to about 45%. In one embodiment, the percent porosity of the lyophilized cake may range from about 20% to about 50%. In one embodiment, the percent porosity of the lyophilized cake may range from about 22% to about 50%. In one embodiment, the percent porosity of the lyophilized cake is about 25% to about 50%. In one embodiment, the percent porosity of the lyophilized cake is from about 30% to about 50%. In one embodiment, the percent porosity of the lyophilized cake is from about 25% to about 45%.

In one embodiment, the amount of surface area of the lyophilized cake may range from about 0.1 m²/g to about 1.1 m²/g, about 0.2 m²/g to about 1.0 m²/g, about 0.3 m²/g to about 0.9 m²/g, about 0.4 m²/g to about 0.8 m²/g, or about 0.5 m²/g to about 0.7 m²/g.

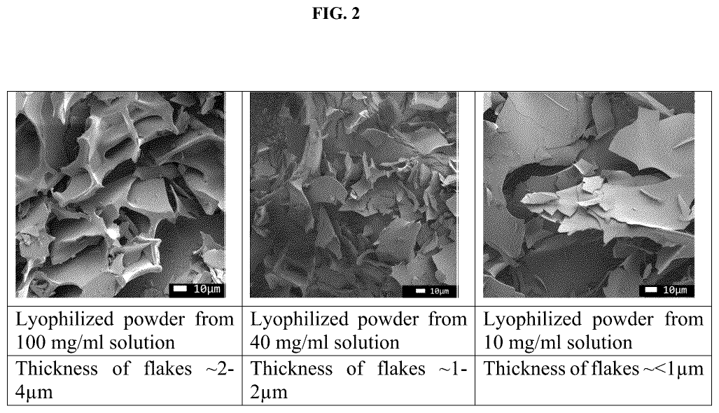

FIG. 2 provides SEM images showing thin flakes and increased surface area per gram of lyophilized phenobarbital sodium powder as the dilution of the lyophilization solution increases.

In one aspect, the present disclosure relates to a lyophilized pharmaceutical composition of phenobarbital or salts thereof prepared by the methods described herein, wherein the lyophilized pharmaceutical composition has flake thickness of not more than about 2 micrometres.

In one aspect, the present disclosure relates to a lyophilized pharmaceutical composition of phenobarbital or salts thereof prepared by the methods described herein, wherein the lyophilized pharmaceutical composition has flake thickness of about 1 micrometres.

In one embodiment, the present invention relates to the lyophilized pharmaceutical composition of phenobarbital sodium having flake thickness of not more than about 2 micrometres. In one embodiment, the lyophilized pharmaceutical composition of phenobarbital sodium has flake thickness of about 2 micrometres, about 1.8 micrometres, about 1.5 micrometres, about 1.2 micrometres, about 1 micrometres, about 0.8 micrometres, or about 0.5 micrometres. Preferably, the flake thickness is about 1 micrometres.

Typically, a single-step freezing batch made with high concentration produces cakes with slight shrinkage in the bottom and coarse-grained irregularity in the middle. This irregularity corresponds to the concentrated core that forms in the freezing stage. The irregularity is eliminated by the two-step freezing. The addition of an annealing step is generally used to obtain a more uniform cake appearance.

In one aspect, the present invention provides methods of lyophilizing a liquid formulation which does not require an annealing step. In the present methods, using low concentration solutions for freezing, the irregularity in cake appearance is minimized or eliminated. For example, phenobarbital sodium particles lyophilised from concentrations ≤6% (w/v) presented microporous surface texture characteristics composed of a microstructure network of particles with visible large pores and cavities under microscopy.

Some of the advantages of the present lyophilization compositions and methods include, for example ease of processing the solution, which simplifies aseptic handling, enhanced stability of the dry powder product, removal of water or solvent without excessive heating of the product, enhanced product stability in a dry state, and rapid and easy dissolution of reconstituted product.

When the lyophiled product is solely made up of an active ingredient without any added excipient, the preparation of active solution concentration becomes critical. The present invention provides methods of preparing a lyophilized pharmaceutical composition of phenobarbital or salts thereof which is can be readily reconstituted and does not have any residual particles. The present inventors have also found that the lyophilization can be performed in absence of annealing in a shorter time without affecting the quality of cake, morphology or reconstitution time.

Typically, the product comprising phenobarbital or salts thereof is used in the strength of 65 mg-130 mg per vial, and is for single use mainly used for starting dose. The 130, 100 or 65 mg per vial doses may contain a significant excess amount of drug product when used for administration of the maintenance dose of e.g., 1.5 mg/kg every 8 hours for a total of 4.5 mg/kg. Further, for maintenance dose, multiple use using same vial, e.g., a 100 mg per vial, is likely to result in waste and may cause contamination and overdosing related errors to the neonatal patient. Hence lower strength vials are required for avoid dosing risk for neonates. To avoid these risks, the present inventors have developed a separate vials containing less product for use in maintenance dose administration. The present lyophilized pharmaceutical compositions provides for such lower strength doses, including 5 mg/vial, 10 mg/vial, 20 mg/vial, 25 mg/vial, 30 mg/vial, 35 mg/vial, 40 mg/vial, 45 mg/vial, 50 mg/vial, 55 mg/vial, 60 mg/vial, 65 mg/vial, 70 mg/vial, 75 mg/vial, 80 mg/vial, 85 mg/vial, 90 mg/vial, 95 mg/vial and 100 mg/vial.

In one embodiment, the lyophilized pharmaceutical composition comprises lyophilized phenobarbital sodium in an amount of 10 mg/vial. In another embodiment, the lyophilized pharmaceutical composition comprises lyophilized phenobarbital sodium in an amount of 100 mg/vial.

In one aspect, the present disclosure provides methods for the treatment of neonatal seizure in newborn infants of 2 weeks of age or younger in need thereof, comprising administering the pharmaceutical compositions described herein.

In one aspect, the present disclosure provides kits for use in the methods described herein. In one embodiment, the kits include multiple packages of the single-dose pharmaceutical composition(s) containing an effective amount of phenobarbital sodium for a single administration in accordance with the methods provided herein. Optionally, diluents necessary for administering the pharmaceutical composition(s) may be included in the kits. For instance, a kit may provide one or more vials containing an amount of phenobarbital sodium that is about 4-5 times the dose in mg/kg indicated for administration in the above methods. Such unit dosage forms preferably contain about 10 mg, about 15 mg, about 50 mg, about 80 mg or about 100 mg.

In one embodiment, the kit may include a loading dose and a a maintainance dose. In one embodiment, the loading dose may include a 100 mg product pack. In one embodiment, the maintanence dose may include a 10 mg product pack.

In one embodiment, the loading dose may include a first loading dose of 20 mg/kg, and a second loading dose if clinically indicated. In one embodiment, the second loading dose may include for a preterm infant 10 mg/kg or 20 mg/kg dose; for a term infant a 20 mg/kg dose; or for 4 Kg neonates, a total dose of 80 mg. In one embodiment, the loading dose may be administered as 1.5 mg/kg every 8 hours or as 2.25 mg/kg every 12 hours, for a total of 4.5 mg/kg.

In one embodiment, the maintainance dose may include 1.5 mg/kg dose administered 3 times a day. In one embodiment, the maintainance dose may include 2.25 mg/kg dose administered 2 times a day. In one embodiment, the maintainance dose may include for 4 Kg neonates, a total dose of 9 mg. In one embodiment, the maintainance dose may be administered for a total duration of 5 days.

In one embodiment, the patient may be administered a single loading dose of 20 mg/kg followed by maintenance dose for five days, wherein the maintenance dose is smaller than the loading dose. In one embodiment, the maintainance dose may include 1.5 mg/kg dose administered 3 times a day. In another embodiment, the maintainance dose may include 2.25 mg/kg dose administered 2 times a day.

In certain embodiments, in addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site. As an example, the kits may include instructions to allow a practitioner (e.g., a physician, nurse, or patient) to administer the composition contained therein to treat a neonatal seizure.

In one embodiment, the kits may include additional components such as instructions or administration schedules for a patient suffering from a disease or condition (e.g., neonatal seizure) to use the pharmaceutical compositions) containing a phenobarbital sodium.

The subject methods and kits can be used to treat neonatal seizure in newborn infants of 2 weeks of age or younger.

As illustrated in the Examples below, the present inventors have observed that the use of certain level of alcohol and specific lyophilization process parameters in the development of the pharmaceutical composition of phenobarbital or slats thereof provides for greatly improved attributes such as stability, quality, morphology and shorter reconstitution time for the resulting product.

### EXAMPLES

### HPLC Method:

HPLC instrument with UV detector or PDA detector;
Reagents/Solvents: Potassium dihydrogen phosphate, Orthophosphoric acid, Acetonitrile, Methanol, Water, Water for Injection;
Needle wash solutions—Water: Methanol (20:80 v/v) & Column wash solution—Water Acetonitrile (50:50 v/v); Preparation of diluted orthophosphoric acid: Dilute 1 ml of concentrated orthophosphoric acid to 10 ml with water;

Preparation of Buffer: Weigh about 2.9 g (2.85 g to 2.95 g) of potassium dihydrogen phosphate and dissolve in 1000 ml of Milli-Q water, adjust pH to 3.5±0.05 with dilute orthophosphoric acid. Filter the solution through 0.45 PVDF Merck Durapore membrane or equivalent filter.

Preparation of Mobile Phase Solutions: Mobile Phase A: Buffer to Acetonitrile (80:20) & Mobile Phase B: Buffer to Acetonitrile (50:50); Diluent: water: methanol (20:80)

### Chromatographic Conditions:

| | |
|---|---|
| Column | Zorbax Eclipse XDB Phenyl, 150 × 4.6 mm, |
| | 5 μm (Agilent) |
| Column temperature | 40° C. |
| Sample Cooler | 10° C. |
| Flow rate | 1.2 ml/min |
| Injection Volume | 10 μl |
| Wavelength | 210 nm (UV/PDA) |
| Run Time | 15 minutes |

| Time (minute) | Mobile phase A % | Mobile phase B % |
|---|---|---|
| 0 | 80 | 20 |
| 4 | 80 | 20 |
| 7 | 20 | 80 |
| 12 | 20 | 80 |
| 12.1 | 80 | 20 |
| 15 | 80 | 20 |

### Example A: Preparation of Phenobarbital Base and Phenobarbital Sodium

The Phenobarbital sodium used herein in the present invention may be prepared according to a general standard method for the preparation of a phenobarbital base and later converting the phenobarbital base to its sodium salt according to general standard methods for the preparation of a salt. The content of ethanol is adjusted to 5000 ppm to 70,000 ppm to meet the objective of the present invention by addition of ethanol. For the purposes of the present invention, the content of ethanol is measured in phenobarbital sodium. More specifically, the content of ethanol can be adjusted in the range of, for example, about 50,000 ppm to about 70,000 ppm or alternatively, from about 12000 to about 25000 ppm.

### Example B: Collapse Temperature of Phenobarbital Sodium

The collapse temperature of phenobarbital sodium sample was determined by cooling the sample to −40° C. at a rate of 10° C./min and holding the sample at this temperature for 5 min before appying vaccum. The frozen sample was dried by gradually raising the temperature from −40° C. to the temperature point when total collapse of the drying structure was observed under the microscope.

The collapse temperature of 40 mg/ml solution of phenobarbital sodium as determined by freeze drying microscope revealed that the onset of the collapse of the drying material began at about −24.4±0.5° C. temperature and total collapse of the drying structure was observed at about −22.8±0.5° C. temperature. Based on this study, collapse temperature of the product in frozen state was concluded as about −24.4±0.5° C.

### Example 1: Lyophilization Cycle

In a clean and dry vessel was taken 800 g of phenobarbital sodium having ethanol content of 66,000 ppm. The phenobarbital sodium was dissolved via stirring in 20,000 ml water for injection and the resulting solution was filtered through PES filter. The vials were filled with 2.5 ml (eq. to 100 mg of phenobarbital sodium) of filtered bulk solution.

Various lyophilization cycles were performed to evaluate the critical stages of lyophilization and achieve the most efficient cycle. Experiments were performed to evaluate the effect of the freezing rate, primary drying temperature, secondary drying temperature, drying times, and pressure on the product.

The optimized lyophilization cycle parameters are described in Table 1 below:

TABLE 1

| | | Temperature | Vacuum | Step | |
|---|---|---|---|---|---|
| Step | | | (micro | Time | |
| No. | Step Type | ° C. | bar) | Hour | Min. |
| 1 | Loading | 5 | — | — | — |
| 2 | Freezing | 5 | — | 00 | 10 |
| 3 | Freezing | −15 | — | 00 | 40 |
| 4 | Freezing | −15 | — | 00 | 30 |
| 5 | Freezing | −45 | — | 00 | 30 |
| 6 | Freezing | −45 | — | 04 | 30 |
| 7 | # Evacuation | — | 133 | 01 | 30 |
| 8 | Drying | −45 | 133 | 00 | 10 |
| 9 | Drying | 20 | 66 | 03 | 00 |
| 10 | Drying | 20 | 66 | 11 | 10 |
| 11 | Drying | 45 | 66 | 00 | 30 |
| 12 | Drying | 45 | 66 | 05 | 00 |
| 13 | Moisture Test | — | 50 | 10 | 60* |
| | (Pressure rise Test) | | | | |
| 14 | Pre aeration | — | — | — | — |
| 15 | Stoppering | — | — | 00 | 01 |
| 16 | Aeration | — | — | — | — |
| 17 | Storage | 25 | — | 00 | 30 |

#The time set point of stoppering step is based on MM:SS
*The hour part of moisture test (PRT) time set point is test repeat time (Minute), minute part is test time (Second)

As illustrated in Table 1, the product was lyophilized by a direct ramp of −45° C. to 20° C. without maintaining primary drying temperature below collapse temperature (i.e., −24.4±0.5° C.) and without any melt-back, collapse or negative impact on critical quality attributes of the product.

### Example 2: Product Characterization

### Product Morphology

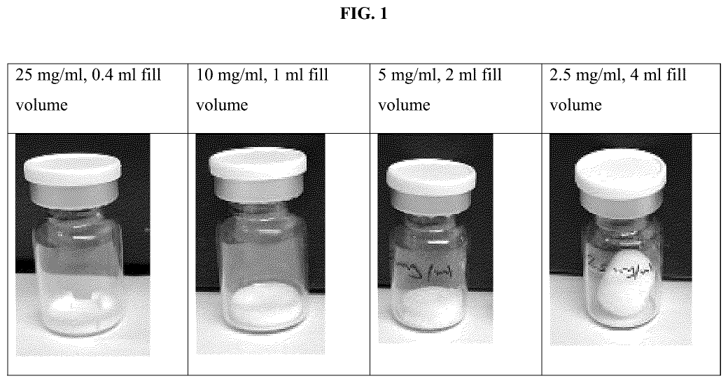

A product was made at lower strength of 10 mg/vial. Vials were filled with 2.5 mg/ml, 5 mg/ml, 10 mg/ml and 25 mg/ml drug solution with a fill volume of 4 ml, 2 ml, 1 ml and 0.4 ml, respectively, and lyophilized. The resultant product for each vial is shown in FIG. 1.

As can be seen from FIG. 1, the morphology of diluted solution is poor at lower concentrations. At 10 mg/ml and 25 mg/ml concentration, good lyophilized cake is formed. On the other hand, for 2.5 mg/ml and 5 mg/ml solutions, the cake shows cotton ball effect due to moisture uptake before stoppering the vial, and blow-off of powder was seen in many vials at these concentrations. It was observed that the obtaining satisfactory morphology for formulation of lower strength could be challenging because for the low dose, adequate cake volume may be needed which is limited by concentration of the solution.

Lyophilized samples were visually inspected for appearance of cake. At lower concentrations of phenobarbital sodium, cake had diffuse appearance due to presence of low solid content that prevented structural rigidity required for the cake formation. At 1 mg/ml concentration, the lyophilized powder loss occurred during secondary lyophilization. Higher concentration showed improved cake formation and structure. Cakes were porous and were easily reconstituted (e.g., less than 5 sec) on addition of distilled water.

### Porosity

2.5 mg/ml to 100 mg/ml of phenobarbital sodium solutions were lyophilized as described in Example 1 it to produce a cake. The blow off of powder was observed to be less than 5 mg/ml. Lyophilized cake was obtained above 10 mg/ml solution. The lyophilized powder was weighed and filled in a 10 ml measuring cylinder. The bulk volume was measured and recorded. Then, the measuring cylinder was tapped on hard surface for 100 times, following which the tapped volume was measured and recorded. From the recorded parameter, bulk density, tapped density void volume and % porosity was calculated using the following equations were used for calculation:

Bulk density=Weight of material/bulk volume

Tapped density=Weight of material/tapped volume

Void volume=Bulk volume−Tapped volume

$$\% porosity = ( 1 - ( tapped volume / bulk volume ) ) \times 100$$

The percent porosity and other attributes of the prepared cakes are provided in Table 2 below:

TABLE 2

| Solution | Fill | Bulk | Bulk | Tapped | Tapped | Void | |
|---|---|---|---|---|---|---|---|
| Strength | volume | Volume | Density | Volume | Density | volume | % |
| (mg/ml) | (ml) | (ml) | (gm/ml) | (ml) | (gm/ml) | (ml) | Porosity |
| 100 | 1 | 1.8 | 0.11 | 1.7 | 0.12 | 0.1 | 5.56 |
| 80 | 1.25 | 1.8 | 0.11 | 1.6 | 0.13 | 0.2 | 11.11 |
| 60 | 1.67 | 2.1 | 0.10 | 1.6 | 0.13 | 0.5 | 23.81 |
| 40 | 2.5 | 2.7 | 0.07 | 2.1 | 0.10 | 0.6 | 22.22 |
| 25 | 4 | 7.4 | 0.02 | 5 | 0.03 | 2.4 | 32.43 |
| 10 | 10 | 8.4 | 0.01 | 4.9 | 0.02 | 3.5 | 41.67 |

The percent porosity was measured using the formula [% porosity=(1−(tapped volume/bulk volume))×100]. Table 2 illustrates that for ranges from about 40% to 24% for the lyophilized cake produced using 10 mg/ml to 60 mg/ml solution. For concentrations above 60 mg/ml, the percent porosity dropped significantly. For example, the lyophilized cake produced using 80 mg/ml and 100 mg/ml solution had a percent porosity of 11.11% and 5.56%, respectively.

Similar results are obtained for phenobarbital sodium having an ethanol content of 12,000-25,000 ppm.

### Reconstituion

A lyophilized vial containing 100 mg cake obtained from different concentrations noted above were reconstituted with 200 microliter water for injection.

Reconstitution time for cake produced by 100 mg/ml drug concentration was observed to be 100 sec, whereas a cake produced by 40 mg/ml drug concentration had reconstitution time of 63 sec.

### Example 3

In a clean and dry flask, 1000 mg of phenobarbital base and 1.67 ml of 96% ethanol were combined and mixed for 1 minute. To this solution, 1.722 ml of the 10% NaOH solution was added and was mixed for 15 minutes. Water for injection sufficient to raise the volume to 25 ml was then added and the resulting solution was mixed for 2 minutes and filtered through a 0.2 micron PES filter.

### Example 4

The procedures of Example 3 were followed, with the exception, that ethanol was not added and the batch size was 600 ml.

The bulk solutions of both the examples were analyzed and the results are shown in Table 3 below:

TABLE 3

| | Example 3 | Example 4 |
|---|---|---|
| | With additional | Without additional |
| | Ethanol | Ethanol |
| Phenobarbital base | Less than 5000 ppm ethanol | |
| Initial analysis | 0M | 0M |
| Total Impurity | 0.06 | 0.314 |
| (%) | | |

Table 3 illustrates that the impurity profile in the composition with additional ethanol (example 3) is better than the composition without ethanol (example 4). It is to be noted that the phenobarbital base API as used in these examples had an ethanol content of less than 5000 ppm. Thus, the composition of example 3 comprises an ethanol content above 5000 ppm. Whereas, the composition of example 4 comprises no additional ethanol and thus the amount of ethanol in the composition of example 4 is equal to the amount present in phenobarbital base API (less than 5000 ppm) or less.

### Example 5

In a clean and dry vessel, 3.64 g phenobarbital base and water for injection were mixed by stirring until the phenobarbital base was dissolved. 6.3 ml of an aqueous 10% NaOH solution was then added while stirring. Water for injection was then added to bring the volume up to 100 ml and the resulting mixture was stirred for 2 minutes.

### Example 6

The procedures of Example 5 were followed, with the exception that following the step of dissolving phenobarbital in water for injection, 6.3 mL of 96% ethanol was added to the mixture. As with Example 5, 6.3 mL of an aqueous 10% NaOH solution was then added while stirring. Water for injection was then added to bring the volume up to 100 ml and the resulting mixture was stirred for 2 minutes.

The results for Examples 5 and 6 are shown in Table 4 below:

TABLE 4

| | |
|---|---|
| Example 5 | Example 6 |
| Without additional ethanol | With additional ethanol |

| | |
|---|---|
| Phenobarbital base | Less than 5000 ppm ethanol |

Total Impurities (%)

| Stage of Analysis | 2-8° C. | 20-25° C. | 2-8° C. | 20-25° C. |
|---|---|---|---|---|
| 0 hr | 0.386 | 0.386 | 0.344 | 0.344 |
| 8 hr | 0.969 | 2.526 | 0.735 | 1.93 |
| 24 hr | 5.065 | 7.423 | 3.982 | 5.586 |

Table 4 illustrates that adding ethanol to the phenobarbital composition in the amounts tested above provided for fewer impurities than the composition that did not have additional ethanol. Ethanol content of 5000 ppm is not sufficient to prevent formation of impurities. The composition wherein additional ethanol was added, afforded a product with improved stability. Degradation of such composition when subjected to stability testing was found to be significantly lower than the composition wherein there was no additional ethanol addition.

### Example 7

In a clean and dry vessel, 4 g of phenobarbital sodium was dissolved via stirring in water for injection. 6.3 ml of 96% ethanol was added to the solution while stirring, and water for injection was then added in an amount sufficient to raise the volume to 100 ml. The resulting solution was stirred for 2 minutes.

### Example 8

The procedures of Example 7 were reproduced, with the exception that the pH of the final solution was adjusted to 9.0 by using 0.1N HCl.

### Example 9

The procedures of Example 7 were reproduced, with the exception that the ethanol was not added.

### Example 10

The procedures of Example 8 were reproduced, with the exception that the ethanol was not added

It is to be noted that the phenobarbital sodium API as used in these examples had an ethanol content of about 66000 ppm.

The results of Examples 7-10 are shown in Table 5 below:

TABLE 5
Composition with Phenobarbital Sodium

| | Example 7 | Example 8 | Example 9 | Example 10 |
|---|---|---|---|---|
| Phenobarbital | About 66000 ppm ethanol | | | |
| Na | 40 mg | 40 mg | 40 mg | 40 mg |
| Ethanol 96% | 0.063 ml | 0.063 ml | — | — |
| pH of Solution | 9.78 | 9.02 | 9.61 | 9.04 |

Total Impurities (%) (2-8° C.)

| | | | | |
|---|---|---|---|---|
| 0 h | 0.004 | 0.005 | 0.007 | 0.005 |
| 1 h | 0.012 | 0.011 | 0.016 | 0.011 |
| 2 h | 0.025 | 0.014 | 0.028 | 0.017 |
| 4 h | 0.026 | 0.017 | 0.031 | 0.018 |
| 8 h | 0.028 | 0.016 | 0.032 | 0.02 |
| 24 h | 0.04 | 0.015 | 0.052 | 0.022 |

Table 5 shows that when the amount of ethanol present in phenobarbital sodium composition was above 5000 ppm then the formation of total impurities was minimized.

### Example 11

In a clean and dry vessel, 60 g of phenobarbital sodium was dissolved via stirring in 1000 ml water for injection and further added 94.5 mL of 96% ethanol. The clarity of the solution was reviewed to ensure that it was clear, and then water for injection sufficient to raise the volume to 1500 ml was added and the solution was stirred followed by filtered through a 0.2 micron PES filter and lyophilization. The lyophilized phenobarbital sodium was kept on stability at 2-8° C. for 36 months. The lyophilized phenobarbital sodium was analyzed for ethanol content at regular intervals.

### Example 12

The procedures of Example 11 were reproduced, with the exception that the pH was adjusted to 9 by the use of 5% HCl and then lyophilized. The lyophilized phenobarbital sodium was kept on stability at 2-8° C. for 36 months. The lyophilized phenobarbital sodium was analyzed for ethanol content at regular intervals.

It is to be noted that the phenobarbital sodium API as used in these examples had an ethanol content of about 66000 ppm.

The results of Examples 11 and 12 are shown in Table 6 below:

TABLE 6

| | Example 11 | Example 12 |
|---|---|---|
| Initial | 38474.7 | 53078.7 |
| 1M | 52188.3 | 66539.3 |
| 3M | 46489.6 | 54457 |
| 6M | 34028.5 | 49584.1 |
| 12M | 37039.5 | 45440 |
| 18M | 42227.5 | 53834.1 |
| 24M | 38035 | 46805 |
| 36M | 45139 | 52043 |

Table 6 shows that when additional ethanol is added in the compositions followed by lyophilizing the solution, the amount of ethanol present in the tested compositions were within the range of from about 34000 ppm to about 66000 ppm.

### Example 13

In a clean and dry vessel, 64 g of phenobarbital sodium was dissolved via stirring in 1200 ml water for injection and the clarity of the solution was observed to ensure that it was clear. Water for injection was then added to the solution in an amount sufficient to raise the volume to 1600 ml and the solution was stirred, filtered through a 0.2 micron PES filter and lyophilized. The lyophilized phenobarbital sodium was kept on stability at 20-25° C. for 36 months. The lyophilized phenobarbital sodium was analyzed for ethanol content at regular intervals.

### Example 14

The procedures of Example 13 were reproduced, with the exception that the batch size was 1800 ml. The lyophilized phenobarbital sodium was kept on stability at 20-25° C. for 36 months.

The lyophilized phenobarbital sodium was analyzed for ethanol content at regular intervals.

It is to be noted that the phenobarbital sodium API as used in these examples had an ethanol content of about 66000 ppm.

The results of Examples 13 and 14 are shown in Table 7 below:

TABLE 7

| | | | | | West-ward's |
|---|---|---|---|---|---|
| | Example 13 | | Example 14 | | Composition* |
| Phenobarbital | About 66000 ppm ethanol | | | | |
| sodium | | | | | |
| Ethanol | — | | | | 0.1 ml/ml |
| Stage of | 24M | 36M | 24M | 36M | analysis after |
| Analysis | | | | | 2.1 year |
| Solvents | 18802 | 16680 | 17030 | 13826 | 66436.1 |
| (Ethanol | | | | | |
| in ppm) | | | | | |
| Unknown | 0.057 | 0.029 | 0.019 | 0.033 | 2.113 |
| Impurities | | | | | |
| (Highest | | | | | |
| Unspecified) | | | | | |
| (%) | | | | | |
| Total | 0.138 | 0.047 | 0.039 | 0.067 | 2.187 |
| Impurities (%) | | | | | |
| Osmolality | 370 | 371 | 361 | 357 | 1341 |
| (mOsm/kg) | | | | | |

*West-ward’s composition is Phenobarbital sodium in ethanol, benzyl alcohol and propylene glycol

Table 7 illustrates that despite the use of a higher level of ethanol in a West-ward's product of phenobarbital sodium at 65 mg/ml, the impurities formation therein is higher than the pharmaceutical composition of preferred embodiments of the present disclosure. Accordingly, the optimum amount of ethanol and lyophilization may be achieved so as to provide for the greatest control of impurities in the composition.

During storage, the amount of ethanol present in the composition may decrease to about 12000 ppm, and thus composition containing ethanol in a range of 12000 to 66000 ppm provide for controlled and lowered levels of impurities. The products of example 13 and 14 were subjected to stability testing. The results of the stability tests are shown in Table 8 below:

TABLE 8

| | Example 13 | Example 14 |
|---|---|---|
| Initial | 24333 | 15510.7 |
| 1M | 18321 | 15639.7 |
| 3M | 22172.2 | 15540 |
| 6M | 23154.3 | 15034 |
| 12M | 22891 | 15018 |
| 18M | 18496 | 12307 |
| 24M | 18802 | 17030 |
| 36M | 16680 | 13826 |

Table 8 shows that the amount of ethanol present in the tested compositions was within the range of about 12000 ppm to about 25000 ppm.

### Examples 15 & 16

In a clean and dry vessel, ˜50 L of water for injection was added and maintained at 2-8° C. Nitrogen purging was performed for 30 min. ˜2.0 kg of phenobarbital sodium was dissolved via stirring for 5-10 min. in to the water for injection and the clarity of the solution was observed to ensure that it was clear. Solution was filtered through a 0.2 micron PES filter and lyophilized. Ethanol content in the lyophilized phenobarbital sodium was measured to be about 13114 ppm. The lyophilized phenobarbital sodium was kept on stability at 20-25° C. for 36 months. Manufactured batch was also evaluated for reconstitution (in-use) stability. Drug product was reconstituted in 0.9% sodium chloride injection and stored at 20-25° C. and 2-8° C. for its stability evaluation. Reconstituted solution was analyzed at predefined time intervals.

It is to be noted that the phenobarbital sodium API as used in these examples had an ethanol content of about 70000 ppm.

The results of examples 15 were as shown in Table 9 below:

TABLE 9

| Storage | 20-25° C. | | | | 2-8° C. | | | |
|---|---|---|---|---|---|---|---|---|
| Stage of analysis | 0 h | 4 h | 8 h | 12 h | 0 h | 12 h | 24 h | 36 h |
| Unknown | BQL | BQL | 0.053 | 0.101 | BQL | 0.072 | BQL | BQL |
| impurity | | | | | | | | |
| (highest | | | | | | | | |
| unspecified) (%) | | | | | | | | |
| Total impurity (%) | BQL | BQL | 0.053 | 0.101 | BQL | 0.072 | BQL | BQL |
| Osmolality | 365 | 371 | 366 | 366 | 371 | 366 | 364 | 368 |
| mOsm/kg | | | | | | | | |

BQL: Below Quantitation Limit (0.05%)

The results of example 16 were as shown in Table 10 below:

TABLE 10

| Storage | 20-25° C. | | | | 2-8° C. | | | |
|---|---|---|---|---|---|---|---|---|
| Stage of analysis | 0 h | 4 h | 8 h | 12 h | 0 h | 12 h | 24 h | 36 h |
| Unknown | BQL | BQL | BQL | 0.088 | BQL | 0.063 | BQL | BQL |
| impurity | | | | | | | | |
| (highest | | | | | | | | |
| unspecified) (%) | | | | | | | | |
| Total impurity (%) | BQL | BQL | BQL | 0.088 | BQL | 0.063 | BQL | BQL |
| Osmolality | 367 | 369 | 369 | 365 | 367 | 367 | 369 | 365 |
| mOsm/kg | | | | | | | | |

BQL: Below Quantitation Limit (0.05%)

### Examples 17 and 18

Two 10% NaOH solutions were prepared in 50 ml flasks by mixing 5 g NaOH with water for injection sufficient to make 30 ml of solution. The resulting solution was mixed and shaken well.

To prepare a bulk solution containing alcohol (example 17), 36.4 mg phenobarbital was placed in a clean and dry vessel. 0.045 ml of a 96% ethanol was added and the resultant was shaken and subject to a vortex for 1 minute. To this solution 0.063 ml of a 10% NaOH solution prepared previously was added and the resultant was subject to a vortex for 15 minutes. Water for injection sufficient to make 1 ml solution was then added and the resultant was subject to a vortex for 10 minutes. The resulting solution was transferred to a measuring cylinder and the pH was checked and adjusted by 0.1N HCl to be 9.78. Water for injection was added to obtain 1 ml of solution and the solution was stirred for 2 minutes. The resulting solution was filtered through a 0.2 micron PES filter, and the filtrate was collected and kept at room temperature (20-25° C.) or 2-8° C. until analysis.

To prepare a bulk solution free from alcohol (example 18), 36.4 mg phenobarbital was placed in a clean and dry vessel. 0.045 ml of a 10% NaOH solution prepared previously was added and the resultant was subject to a vortex for 15 minutes. Water for injection sufficient to make 1 ml solution was then added and the resultant was subject to a vortex for 10 minutes. The resulting solution was transferred to a measuring cylinder and the pH was checked and adjusted by 0.1N HCl to be 9.74. Water for injection was added to obtain 1 ml of solution and the solution was stirred for 2 minutes. The resulting solution was filtered through a 0.2 micron PES filter, and the filtrate was collected and kept at room temperature (20-25° C.) or 2-8° C. until analysis.

The two bulk solutions were analyzed at set time periods and Table 11 shows the results.

TABLE 11

| | | 20-25° C. | | | | 2-8° C. | | |
|---|---|---|---|---|---|---|---|---|
| | Impurity | 0 h | 12 h | 24 h | 36 h | 0 h | 24 h | 36 h |
| Example | Unknown | 0.042 | 0.371 | 0.708 | 1.002 | 0.042 | 0.151 | 0.219 |
| 17 | impurity | | | | | | | |
| | (highest | | | | | | | |
| | unspecified) | | | | | | | |
| | (%) | | | | | | | |
| | Total | 0.064 | 0.398 | 0.764 | 1.091 | 0.064 | 0.173 | 0.236 |
| | Impurity | | | | | | | |
| | (%) | | | | | | | |
| Example | Unknown | 0.065 | 0.634 | 1.207 | 1.724 | 0.065 | 0.269 | 0.384 |
| 18 | impurity | | | | | | | |
| | (highest | | | | | | | |
| | unspecified) | | | | | | | |
| | (%) | | | | | | | |
| | Total | 0.078 | 0.674 | 1.288 | 1.859 | 0.078 | 0.293 | 0.404 |
| | Impurity | | | | | | | |
| | (%) | | | | | | | |

Even though certain specific embodiments are thoroughly described in the present application, it should be understood that the same concepts disclosed with respect to those specific embodiments are also applicable to other embodiments. Furthermore, individual elements of the compositions and methods disclosed herein are described with reference to particular embodiments only for the sake of convenience. It should be understood that individual elements of the compositions and methods disclosed herein are applicable to embodiments other than the specific embodiments in which they are described.

In addition, it should be understood that the scope of the present disclosure is not limited to the above-described embodiments, and those skilled in the art will appreciate that various modifications and alterations are possible without departing from the scope of the present disclosure. For example, the batch sizes may be altered by a person having ordinary skill in the art while staying within the present disclosure.

Recitation of ranges of values herein are merely intended to serve as a shorthand method of referring individually to each separate value falling within the range, unless otherwise indicated herein, and each separate value is incorporated into the specification as if it were individually recited herein, as well as values in between the recited values. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated.

### FEATURES OF THE INVENTION

A1. A pharmaceutical composition of phenobarbital or salts thereof that has been reconstituted from a lyophilized pharmaceutical composition of phenobarbital or salts thereof, wherein the amount of total impurities in the pharmaceutical composition does not exceed 0.2% following 12 hours of storage at 20-25° C. or 36 hours of storage at 2-8° C.
A2. The pharmaceutical composition of feature A1, wherein the pharmaceutical composition has an alcohol content in the range from about 5000 ppm to about 66000 ppm or alternatively from about 5000 ppm to about 70000 ppm.
A3. The pharmaceutical composition of feature A1, wherein the pharmaceutical composition has an alcohol content is in the range from about 12000 ppm to about 66000 ppm.
A4. The pharmaceutical composition of feature A1, wherein the pharmaceutical composition has an alcohol content is in the range from about 12000 ppm to about 25000 ppm.
A5. The pharmaceutical composition of features A2-4, wherein alcohol is a C₁-C₃ alcohol, such as ethanol.
A6. The pharmaceutical composition of feature A1, wherein phenobarbital or salts thereof is phenobarbital sodium.
A7. The pharmaceutical composition of feature A1, wherein the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.
A8. The pharmaceutical composition of feature A1, wherein the lyophilized pharmaceutical composition is reconstituted with water for injection, an aqueous saline or an aqueous dextrose solution.
A9. The pharmaceutical composition of feature A1, wherein the pharmaceutical composition is an aqueous solution for injection of phenobarbital or salts thereof.
A10. The pharmaceutical composition of feature A1, wherein phenobarbital or salts thereof is present in a concentration of 10-200 mg/ml.
A11. The pharmaceutical composition of feature A1, wherein the pharmaceutical composition is free of benzyl alcohol.
A12. The pharmaceutical composition of feature A1, wherein the pharmaceutical composition is free of propylene glycol.
A13. The pharmaceutical composition of feature A1, wherein the pharmaceutical composition has an osmolality below 500 mOsm/kg.
A14. A method for the treatment of neonatal seizure in newborn infants of 2 weeks of age or younger in need thereof, comprising administering the pharmaceutical composition of phenobarbital or salts thereof that has been reconstituted from a lyophilized pharmaceutical composition of phenobarbital or salts thereof, wherein the amount of total impurities in the pharmaceutical composition does not exceed 0.2% following 12 hours of storage at 20-25° C. or 36 hours of storage at 2-8° C.
A15. The method of feature A14, wherein the pharmaceutical composition is administered intravenously by infusion at a dose of 20 mg/kg over a course of 15 minutes.
A16. The method of feature A14, wherein the method comprises administering the pharmaceutical composition at an initial loading dose of 20 mg/kg over a course of 15 minutes and measuring the electrographic seizures; and if electrographic seizures persist or recur after completion of the initial loading dose, a second dose of 20 mg/kg is administered over subsequent 15 minutes for a total loading dose of 40 mg/kg.
A17. The method of feature A14, wherein the method comprises administering the pharmaceutical composition to neonates in whom correctable abnormalities have been excluded or corrected.
A18. The method of feature A17, wherein the correctable abnormalities are selected from hypoglycemia or hypocalcemia.
A19. The method of feature A14, wherein the pharmaceutical composition has an alcohol content in the range from about 5000 ppm to about 66000 ppm or alternatively from about 5000 ppm to about 70000 ppm.
A20. The method of feature A14, wherein the pharmaceutical composition has an alcohol content is in the range from about 12000 ppm to about 66000 ppm.
A21. The method of feature A14, wherein the pharmaceutical composition has an alcohol content is in the range from about 12000 ppm to about 25000 ppm.
A22. The method of features A19-21, wherein alcohol is a C₁-C₃ alcohol, such as ethanol.
A23. The method of feature A14, wherein phenobarbital or salts thereof is phenobarbital sodium.
A24. The method of feature A14, wherein the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.
A25. The method of feature A14, wherein the lyophilized pharmaceutical composition is reconstituted with water for injection, an aqueous saline or an aqueous dextrose solution.
A26. The method of feature A14, wherein the pharmaceutical composition is an aqueous solution for injection of phenobarbital or salts thereof.
A27. The method of feature A14, wherein phenobarbital or salts thereof is present in a concentration of 10-200 mg/ml.
A28. The method of feature A14, wherein the pharmaceutical composition is free of benzyl alcohol.
A29. The method of feature A14, wherein the pharmaceutical composition is free of propylene glycol.
A30. The method of feature A14, wherein the pharmaceutical composition has an osmolality below 500 mOsm/kg.
A31. A pharmaceutical composition of phenobarbital or salts thereof, wherein the pharmaceutical composition has an alcohol content in the range from about 5000 ppm to about 66000 ppm or alternatively from about 5000 ppm to about 70000 ppm.
A32. The pharmaceutical composition of feature A31, wherein the composition has an alcohol content in the range from about 12000 ppm to about 66000 ppm.
A33. The pharmaceutical composition of feature A31, wherein the composition has an alcohol content in the range from about 12000 ppm to about 25000 ppm.
A34. The pharmaceutical composition of features A31-33, wherein alcohol is a C₁-C₃ alcohol, such as ethanol.
A35. The pharmaceutical composition of feature A31, wherein phenobarbital or salts thereof is phenobarbital sodium.
A36. The pharmaceutical composition of feature A31, wherein the pharmaceutical composition is a lyophilized pharmaceutical composition of phenobarbital or salts thereof.
A37. The pharmaceutical composition of feature A36, wherein the lyophilized pharmaceutical composition is stable up to 36 months of storage at 20-25° C.
A38. The pharmaceutical composition of feature A37, wherein the amount of total impurities present following 36 months of storage at 20-25° C. does not exceed 0.5%.
A39. The pharmaceutical composition of feature A37, wherein the amount of total impurities present following 36 months of storage at 20-25° C. does not exceed 0.2%.
A40. The pharmaceutical composition of feature A31, wherein the pharmaceutical composition is an aqueous solution for injection of phenobarbital or salts thereof.
A41. The pharmaceutical composition of feature A40, wherein the aqueous solution is reconstituted from the lyophilized pharmaceutical composition of phenobarbital or salts thereof.
A42. The pharmaceutical composition of feature A41, wherein the lyophilized pharmaceutical composition of phenobarbital or salts thereof is reconstituted with water for injection, an aqueous saline or an aqueous dextrose solution.
A43. The pharmaceutical composition of feature A40, wherein phenobarbital or salts thereof is present in a concentration of 10-200 mg/ml.
A44. The pharmaceutical composition of feature A40, wherein the aqueous solution is stable up to 12 hours of storage at 20-25° C.
A45. The pharmaceutical composition of feature A44, wherein the amount of total impurities present following 12 hours of storage at 20-25° C. does not exceed 0.5%.
A46. The pharmaceutical composition of feature A44, wherein the amount of total impurities present following 12 hours of storage at 20-25° C. does not exceed 0.2%.
A47. The pharmaceutical composition of feature A40, wherein the aqueous solution is stable up to 36 hours of storage at 2-8° C.
A48. The pharmaceutical composition of feature A47, wherein the amount of total impurities present following 36 hours of storage at 2-8° C. does not exceed 0.5%.
A49. The pharmaceutical composition of feature A47, wherein the amount of total impurities present following 36 hours of storage at 2-8° C. does not exceed 0.2%.
A50. The pharmaceutical composition of feature A40, wherein the aqueous solution has an osmolality below 500 mOsm/kg.
A51. The pharmaceutical composition of features A38-39, A45-46 and A48-49, wherein the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.
A52. The pharmaceutical composition of feature A31, wherein the pharmaceutical composition is free of benzyl alcohol.
A53. The pharmaceutical composition of feature A31, wherein the pharmaceutical composition is free of propylene glycol.
A54. A method for the treatment of neonatal seizure in newborn infants of 2 weeks of age or younger in need thereof, comprising administering the pharmaceutical composition of phenobarbital or salts thereof, wherein the pharmaceutical composition has an alcohol content in the range from about 5000 ppm to about 66000 ppm or alternatively from about 5000 ppm to about 70000 ppm.
A55. The method of feature A54, wherein the pharmaceutical composition is administered intravenously by infusion at a dose of 20 mg/kg over a course of 15 minutes.
A56. The method of feature A54, wherein the method comprises administering the pharmaceutical composition at an initial loading dose of 20 mg/kg over a course of 15 minutes and measuring the electrographic seizures; and if the electrographic seizures persist or recur after completion of the initial loading dose, a second dose 20 mg/kg is administered over the subsequent 15 minutes for a total loading dose of 40 mg/kg.
A57. The method of feature A54, wherein the method comprises administering the pharmaceutical composition to neonates in whom correctable abnormalities have been excluded or corrected.
A58. The method of feature A57, wherein the correctable abnormalities are selected from hypoglycemia or hypocalcemia.
A59. The method of feature A54, wherein the pharmaceutical composition has an alcohol content in the range from about 12000 ppm to about 66000 ppm.
A60. The method of feature A54, wherein the pharmaceutical composition has an alcohol content in the range from about 12000 ppm to about 25000 ppm.
A61. The method of features A54, A59-60, wherein alcohol is a C₁-C₃ alcohol, such as ethanol.
A62. The method of feature A54, wherein phenobarbital or salts thereof is phenobarbital sodium.
A63. The method of feature A54, wherein the pharmaceutical composition is a lyophilized pharmaceutical composition of phenobarbital or salts thereof.
A64. The method of features A63, wherein the lyophilized pharmaceutical composition is stable up to 36 months of storage at 20-25° C.
A65. The method of features A64, wherein the amount of total impurities present following 36 months of storage at 20-25° C. does not exceed 0.5%.
A66. The method of features A64, wherein the amount of total impurities present following 36 months of storage at 20-25° C. does not exceed 0.2%.
A67. The method of feature A54, wherein the pharmaceutical composition is an aqueous solution for injection of phenobarbital or salts thereof.
A68. The method of features A67, wherein the aqueous solution is reconstituted from the lyophilized pharmaceutical composition of phenobarbital or salts thereof.
A69. The method of features A68, wherein the lyophilized pharmaceutical composition is reconstituted with water for injection, an aqueous saline or an aqueous dextrose solution.
A70. The method of features A67, wherein the aqueous solution is stable up to 12 hours of storage at 20-25° C.
A71. The method of features A70, wherein the amount of total impurities present following 12 hours of storage at 20-25° C. does not exceed 0.2%.
A72. The method of features A67, wherein the aqueous solution is stable up to 36 hours of storage at 2-8° C.
A73. The method of features A72, wherein the amount of total impurities present following 36 hours of storage at 2-8° C. does not exceed 0.2%.
A74. The method of features A67, wherein the aqueous solution has an osmolality below 500 mOsm/kg.
A75. The method of feature A54, wherein the pharmaceutical composition is free of benzyl alcohol.
A76. The method of feature A54, wherein the pharmaceutical composition is free of propylene glycol.
A77. A lyophilized pharmaceutical composition of phenobarbital or salts thereof, wherein the composition has an alcohol content in the range from about 5000 ppm to about 66000 ppm or alternatively from about 5000 ppm to about 70000 ppm.
A78. The lyophilized pharmaceutical composition of feature A77, wherein the lyophilized pharmaceutical composition has an alcohol content in the range from about 12000 ppm to about 66000 ppm.
A79. The lyophilized pharmaceutical composition of feature A77, wherein the lyophilized pharmaceutical composition has an alcohol content in the range from about 12000 ppm to about 25000 ppm.
A80. The lyophilized pharmaceutical composition of features A77-79, wherein alcohol is a C₁-C₃ alcohol, such as ethanol.
A81. The lyophilized pharmaceutical composition of feature A77, wherein phenobarbital or salts thereof is phenobarbital sodium.
A82. The lyophilized pharmaceutical composition of feature A77 is stable up to 36 months of storage at 20-25° C.
A83. The lyophilized pharmaceutical composition of feature A82, wherein the amount of total impurities present following 36 months of storage at 20-25° C. does not exceed 0.5%.
A84. The lyophilized pharmaceutical composition of feature A82, wherein the amount of total impurities present following 36 months of storage at 20-25° C. does not exceed 0.2%.
A85. The lyophilized pharmaceutical composition of features A83-84, wherein the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.
A86. The lyophilized pharmaceutical composition of feature A77, wherein the lyophilized pharmaceutical composition is free of benzyl alcohol.
A87. The lyophilized pharmaceutical composition of feature A77, wherein the lyophilized pharmaceutical composition is free of propylene glycol.
A88. A method for the treatment of neonatal seizure in newborn infants of 2 weeks of age or younger in need thereof, comprising administering the lyophilized pharmaceutical composition of phenobarbital or salts thereof, and wherein the lyophilized pharmaceutical composition has an alcohol content in the range from about 5000 ppm to about 66000 ppm or alternatively from about 5000 ppm to about 70000 ppm.
A89. The method of feature A88 comprises reconstituting the lyophilized pharmaceutical composition of phenobarbital or salts thereof immediately prior to the administration.
A90. The method of feature A89, wherein the lyophilized pharmaceutical composition is reconstituted with water for injection, an aqueous saline or an aqueous dextrose solution to obtain the aqueous solution for injection of phenobarbital or salts thereof.
A91. The method of feature A90, wherein the aqueous solution is administered intravenously by infusion at a dose of 20 mg/kg over a course of 15 minutes.
A92. The method of features A88 and A89, wherein the method comprises administering the aqueous solution at an initial loading dose of 20 mg/kg over a course of 15 minutes and measuring the electrographic seizures; and if the electrographic seizures persist or recur after completion of the initial loading dose, a second dose 20 mg/kg is administered over the subsequent 15 minutes for a total loading dose of 40 mg/kg.
A93. The method of feature A88, wherein the method comprises administering to neonates in whom correctable abnormalities have been excluded or corrected.
A94. The method of feature A93, wherein the correctable abnormalities are selected from hypoglycemia or hypocalcemia.
A95. The method of feature A88, wherein the lyophilized pharmaceutical composition has an alcohol content in the range from about 12000 ppm to about 66000 ppm.
A96. The method of feature A88, wherein the lyophilized pharmaceutical composition has an alcohol content in the range from about 12000 ppm to about 25000 ppm.
A97. The method of features A88, A95-96, wherein alcohol is a C₁-C₃ alcohol, such as ethanol.
A98. The method of feature A88, wherein phenobarbital or salts thereof is phenobarbital sodium.
A99. The method of feature A88, wherein the lyophilized pharmaceutical composition is stable up to 36 months of storage at 20-25° C.
A100. The method of feature A99, wherein the amount of total impurities present following 36 months of storage at 20-25° C. does not exceed 0.5%.
A101. The method of feature A99, wherein the amount of total impurities present following 36 months of storage at 20-25° C. does not exceed 0.2%.
A102. The method of features A100-101, wherein the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.
A103. The method of feature A88, wherein the lyophilized pharmaceutical composition is free of benzyl alcohol.
A104. The method of feature A88, wherein the lyophilized pharmaceutical composition is free of propylene glycol.
A105. A pharmaceutical composition of phenobarbital or salts thereof that has been reconstituted from a lyophilized pharmaceutical composition of phenobarbital or salts thereof, wherein the pharmaceutical composition has an alcohol content in the range from about 5000 ppm to about 66000 ppm or alternatively from about 5000 ppm to about 70000 ppm.
A106. The pharmaceutical composition of feature A105, wherein the pharmaceutical composition has an alcohol content in the range from about 12000 ppm to about 66000 ppm.
A107. The pharmaceutical composition of feature A105, wherein the pharmaceutical composition has an alcohol content in the range from about 12000 ppm to about 25000 ppm.
A108. The pharmaceutical composition of feature A105-107, wherein alcohol is a C₁-C₃ alcohol, such as ethanol.
A109. The pharmaceutical composition of feature A105, wherein phenobarbital or salts thereof is phenobarbital sodium.
A110. The pharmaceutical composition of feature A105, wherein the lyophilized pharmaceutical composition is reconstituted with water for injection, an aqueous saline or an aqueous dextrose solution.

A111. The pharmaceutical composition of feature A105 is an aqueous solution for injection of phenobarbital or salts thereof.
A112. The pharmaceutical composition of feature A105, wherein phenobarbital or salts thereof is present in a concentration of 10-200 mg/ml.
A113. The pharmaceutical composition of feature A105, wherein the pharmaceutical composition is stable up to 12 hours of storage at 20-25° C.
A114. The pharmaceutical composition of feature A113, wherein the amount of total impurities present following 12 hours of storage at 20-25° C. does not exceed 0.5%.
A115. The pharmaceutical composition of feature A113, wherein the amount of total impurities present following 12 hours of storage at 20-25° C. does not exceed 0.2%.
A116. The pharmaceutical composition of feature A105, wherein the pharmaceutical composition is stable up to 36 hours of storage at 2-8° C.
A117. The pharmaceutical composition of feature A116, wherein the amount of total impurities present following 36 hours of storage at 2-8° C. does not exceed 0.5%.
A118. The pharmaceutical composition of feature A116, wherein the amount of total impurities present following 36 hours of storage at 2-8° C. does not exceed 0.2%.
A119. The pharmaceutical composition of features A114-115 and A117-118, wherein the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.
A120. The pharmaceutical composition of feature A105, wherein the pharmaceutical composition has an osmolality below 500 mOsm/kg.
A121. The pharmaceutical composition of feature A105, wherein the pharmaceutical composition is free of benzyl alcohol.
A122. The pharmaceutical composition of feature AA105, wherein the pharmaceutical composition is free of propylene glycol.
A123. A method for the treatment of neonatal seizure in newborn infants of 2 weeks of age or younger in need thereof, comprising administering a pharmaceutical composition of phenobarbital or salts thereof that has been reconstituted from a lyophilized pharmaceutical composition of phenobarbital or salts thereof, and wherein the pharmaceutical composition has an alcohol content in the range from about 5000 ppm to about 66000 ppm or alternatively from about 5000 ppm to about 70000 ppm.
A124. The method of feature A123, wherein the pharmaceutical composition is administered intravenously by infusion at a dose of 20 mg/kg over a course of 15 minutes.
A125. The method of feature A123, wherein the method comprises administering the pharmaceutical composition at an initial loading dose of 20 mg/kg over a course of 15 minutes and measuring the electrographic seizures; and if the electrographic seizures persist or recur after completion of the initial loading dose, a second dose 20 mg/kg is administered over the subsequent 15 minutes for a total loading dose of 40 mg/kg.
A126. The method of feature A123, wherein the method comprises administering the pharmaceutical composition to neonates in whom correctable abnormalities have been excluded or corrected.
A127. The method of feature A126, wherein the correctable abnormalities are selected from hypoglycemia or hypocalcemia.
A128. The method of feature A123, wherein the pharmaceutical composition has an alcohol content in the range from about 12000 ppm to about 66000 ppm.
A129. The method of feature A126, wherein the pharmaceutical composition has an alcohol content in the range from about 12000 ppm to about 25000 ppm.
A130. The method of features A123, A128-129, wherein alcohol is a C₁-C₃ alcohol, such as ethanol.
A131. The method of feature A123, wherein phenobarbital or salts thereof is phenobarbital sodium.
A132. The method of feature A123, wherein the lyophilized pharmaceutical composition is reconstituted with water for injection, an aqueous saline or an aqueous dextrose solution.
A133. The method of feature A123, wherein the pharmaceutical composition is an aqueous solution for injection of phenobarbital or salts thereof.
A134. The method of feature A123, wherein phenobarbital or salts thereof is present in a concentration of 10-200 mg/ml.
A135. The method of feature A123, wherein the pharmaceutical composition is stable up to 12 hours of storage at 20-25° C.
A136. The method of feature A135, wherein the amount of total impurities present following 12 hours of storage at 20-25° C. does not exceed 0.2%.
A137. The method of feature A123, wherein the pharmaceutical composition is stable up to 36 hours of storage at 2-8° C.
A138. The method of feature A137, wherein the amount of total impurities present following 36 hours of storage at 2-8° C. does not exceed 0.2%.
A139. The method of features A136 and A138, wherein the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.
A140. The method of feature A123, wherein the pharmaceutical composition has an osmolality below 500 mOsm/kg.
A141. The method of feature A123, wherein the pharmaceutical composition is free of benzyl alcohol.
A142. The method of feature A123, wherein the pharmaceutical composition is free of propylene glycol.
A143. A process of preparing the lyophilized pharmaceutical composition of phenobarbital or salts thereof having an alcohol content in the range from about 5000 ppm to about 66000 ppm or alternatively from about 5000 ppm to about 70000 ppm, wherein the process comprises dissolving phenobarbital or salts thereof in water to obtain an aqueous solution of phenobarbital or salts thereof in a concentration of 10-200 mg/ml and lyophilizing the aqueous solution to obtain lyophilized pharmaceutical composition of phenobarbital or salts thereof.
A144. The process of feature A143, wherein the process comprises measuring the alcohol content of the aqueous solution of phenobarbital or salts thereof and if the alcohol content is below 5000 ppm then the process further comprises a step of adding alcohol to achieve the alcohol content of at least about 5000 ppm.
A145. The process of features A143, wherein the process comprises measuring the alcohol content of the lyophilized pharmaceutical composition of phenobarbital or salts thereof and if the alcohol content is above 66000 ppm or about 70000 ppm (whichever is desired), the process further comprises repeating the lyophilization step multiple times till the alcohol content of the lyophilized pharmaceutical composition is not more than about 66000 ppm or about 70000 ppm.
A146. The process of feature A143 further comprises addition of a pH modifier to the aqueous solution of phenobarbital or salts thereof to achieve a pH in a range of 9-10.5.
A147. The process of feature A143, wherein the lyophilized pharmaceutical composition has an alcohol content in the range from about 12000 ppm to about 66000 ppm.
A148. The process of feature A143, wherein the lyophilized pharmaceutical composition has an alcohol content in the range from about 12000 ppm to about 25000 ppm.
A149. The process of features A143, A147-148, wherein alcohol is a C₁-C₃ alcohol, such as ethanol.
A150. The process of feature A143, wherein phenobarbital or salts thereof is phenobarbital sodium.
A151. The process of feature A143, wherein the lyophilized pharmaceutical composition is stable up to 36 months of storage at 20-25° C.
A152. The process of feature A151, wherein the amount of total impurities present following 36 months of storage at 20-25° C. does not exceed 0.5%.
A153. The process of feature A151, wherein the amount of total impurities present following 36 months of storage at 20-25° C. does not exceed 0.2%.
A154. The process of features A152-153, wherein the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.
A155. The process of feature A143, wherein the lyophilized pharmaceutical composition is free of benzyl alcohol.
A156. The process of feature A143, wherein the lyophilized pharmaceutical composition is free of propylene glycol.
A157. A process of preparing the lyophilized pharmaceutical composition of phenobarbital or salts thereof having an alcohol content in the range from about 5000 ppm to about 66000 ppm or alternatively from about 5000 ppm to about 70000 ppm, the process comprises dissolving phenobarbital or salts thereof in water to obtain an aqueous solution having a concentration 10-200 mg/ml; measuring the alcohol content of aqueous solution; if the alcohol content is below 5000 ppm, adding an alcohol to achieve the alcohol content of at least about 5000 ppm; lyophilizing the aqueous solution to obtain lyophilized pharmaceutical composition; measuring the alcohol content of the lyophilized pharmaceutical composition; if the alcohol content is above about 66000 ppm or above about 70000 ppm (whichever is desired), repeating the lyophilization step multiple times till the alcohol content of the lyophilized pharmaceutical composition is not more than about 66000 ppm or about 70000 ppm.
A158. The process of feature A157 further comprises addition of a pH modifier to the aqueous solution of phenobarbital or salts thereof to achieve a pH in a range of 9-10.5.
A159. The process of feature A157, wherein the lyophilized pharmaceutical composition has an alcohol content in the range from about 12000 ppm to about 66000 ppm.
A160. The process of feature A157, wherein the lyophilized pharmaceutical composition has an alcohol content in the range from about 12000 ppm to about 25000 ppm.
A161. The process of features A157, A159-160, wherein alcohol is a C₁-C₃ alcohol, such as ethanol.
A162. The process of feature A157, wherein phenobarbital or salts thereof is phenobarbital sodium.
A163. The process of feature A157, wherein the lyophilized pharmaceutical composition is stable up to 36 months of storage at 20-25° C.
A164. The process of feature A163, wherein the amount of total impurities present following 36 months of storage at 20-25° C. does not exceed 0.5%.
A165. The process of feature A163, wherein the amount of total impurities present following 36 months of storage at 20-25° C. does not exceed 0.2%.
A166. The process of features A164-165, wherein the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.
A167. The process of feature A157, wherein the lyophilized pharmaceutical composition is free of benzyl alcohol.
A168. The process of feature A157, wherein the lyophilized pharmaceutical composition is free of propylene glycol.
A169. A process of preparing the pharmaceutical composition of phenobarbital or salts thereof that has been reconstituted from a lyophilized pharmaceutical composition of phenobarbital or salts thereof, wherein the pharmaceutical composition has an alcohol content in the range from about 5000 ppm to about 66000 ppm or alternatively from about 5000 ppm to about 70000 ppm, the process comprises dissolving phenobarbital or salts thereof in water to obtain an aqueous solution having a concentration 10-200 mg/ml; lyophilizing the aqueous solution to obtain lyophilized pharmaceutical composition of phenobarbital or salts thereof, and reconstituting the lyophilized pharmaceutical composition to obtain the pharmaceutical composition of phenobarbital or salts thereof.
A170. The process of feature A169, wherein the process comprises measuring the alcohol content of the aqueous solution of phenobarbital or salts thereof and if the alcohol content is below 5000 ppm then the process further comprises a step of adding alcohol to achieve the alcohol content of at least about 5000 ppm.
A171. The process of features A169, wherein said process comprises measuring the alcohol content of the lyophilized pharmaceutical composition of phenobarbital or salts thereof and if the alcohol content is above about 66000 ppm or about 70000 ppm (whichever is desired), the process further comprises repeating the lyophilization step multiple times till the alcohol content of the lyophilized pharmaceutical composition is not more than about 66000 ppm or about 70000 ppm.
A172. The process of feature A169 further comprises addition of pH modifier to the aqueous solution of phenobarbital or salts thereof to achieve a pH in a range of 9-10.5.
A173. The process of feature A169, wherein the lyophilized pharmaceutical composition is reconstituted with water for injection, an aqueous saline or an aqueous dextrose solution.
A174. The process of feature A169, wherein the pharmaceutical composition is an aqueous solution for injection of phenobarbital or salts thereof.
A175. The process of feature A169, wherein the pharmaceutical composition has an alcohol content in the range from about 12000 ppm to about 66000 ppm.
A176. The process of feature A169, wherein the pharmaceutical composition has an alcohol content in the range from about 12000 ppm to about 25000 ppm.
A177. The process of feature A169, A175-176, wherein alcohol is a C₁-C₃ alcohol, such as ethanol.
A178. The process of feature A169, wherein phenobarbital or salts thereof is phenobarbital sodium.
A179. The process of feature A169, wherein the pharmaceutical composition is stable up to 12 hours of storage at 20-25° C.
A180. The process of feature A179, wherein the amount of total impurities present following 12 hours of storage at 20-25° C. does not exceed 0.2%.
A181. The process of feature A169, wherein the pharmaceutical composition is stable up to 36 hours of storage at 2-8° C.
A182. The process of feature A181, wherein the amount of total impurities present following 36 hours of storage at 2-8° C. does not exceed 0.2%.
A183. The process of features A180 and A182, wherein the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.
A184. The process of feature A169, wherein the pharmaceutical composition has an osmolality below 500 mOsm/kg.
A185. The process of feature A169, wherein the pharmaceutical composition is free of benzyl alcohol.
A186. The process of feature A169, wherein the pharmaceutical composition is free of propylene glycol.
A187. A process of preparing the pharmaceutical composition of phenobarbital or salts thereof that has been reconstituted from a lyophilized pharmaceutical composition of phenobarbital or salts thereof, wherein the pharmaceutical composition has an alcohol content in the range from about 5000 ppm to about 66000 ppm or alternatively from about 5000 ppm to about 70000 ppm, wherein the process comprises dissolving phenobarbital or salts thereof in water to obtain an aqueous solution having a concentration 10-200 mg/ml; measuring the alcohol content of aqueous solution; if the alcohol content is below 5000 ppm, adding an alcohol to achieve the alcohol content of at least about 5000 ppm; lyophilizing the aqueous solution to obtain lyophilized pharmaceutical composition; measuring the alcohol content of the lyophilized pharmaceutical composition; if the alcohol content is above 66000 ppm, repeating the lyophilization step multiple times till the alcohol content of the lyophilized pharmaceutical composition is not more than about 66000 ppm or not more than 70000 ppm (whichever is desired); and reconstituting the lyophilized pharmaceutical composition to obtain the pharmaceutical composition of phenobarbital or salts thereof.
A188. The process of feature A187 further comprises addition of pH modifier to the aqueous solution of phenobarbital or salts thereof to achieve a pH in a range of 9-10.5.
A189. The process of feature A187, wherein the lyophilized pharmaceutical composition is reconstituted with water for injection, an aqueous saline or an aqueous dextrose solution.
A190. The process of feature A187, wherein the pharmaceutical composition is an aqueous solution for injection of phenobarbital or salts thereof.
A191. The process of feature A187, wherein the pharmaceutical composition has an alcohol content in the range from about 12000 ppm to about 66000 ppm.
A192. The process of feature A187, wherein the pharmaceutical composition has an alcohol content in the range from about 12000 ppm to about 25000 ppm.
A193. The process of feature A187, A191-192, wherein alcohol is a C₁-C₃ alcohol, such as ethanol.
A194. The process of feature A187, wherein phenobarbital or salts thereof is phenobarbital sodium.
A195. The process of feature A187, wherein the pharmaceutical composition is stable up to 12 hours of storage at 20-25° C.
A196. The process of feature A195, wherein the amount of total impurities present following 12 hours of storage at 20-25° C. does not exceed 0.2%.
A197. The process of feature A187, wherein the pharmaceutical composition is stable up to 36 hours of storage at 2-8° C.
A198. The process of feature A197, wherein the amount of total impurities present following 36 hours of storage at 2-8° C. does not exceed 0.2%.
A199. The process of features A196 and A198, wherein the total impurities are selected from 2-phenyl-2-ethyl acetyl urea, 2-phenyl-2-ethyl-malonamide, α-phenylbutyrylguanidine, 2-phenylbutyric acid and 5-methyl-5-phenylbarbituric acid.
A200. The process of feature A187, wherein the pharmaceutical composition has an osmolality below 500 mOsm/kg.
A201. The process of feature A187, wherein the pharmaceutical composition is free of benzyl alcohol.
A202. The process of feature A187, wherein the pharmaceutical composition is free of propylene glycol.
A203. A lyophilized pharmaceutical composition of phenobarbital or salts thereof having an alcohol content in the range from about 5000 ppm to about 66000 ppm, wherein the lyophilized pharmaceutical composition is obtained by a process comprising: dissolving phenobarbital or salts thereof in water to obtain an aqueous solution having a concentration 10-200 mg/ml and lyophilizing the aqueous solution to obtain lyophilized pharmaceutical composition of phenobarbital or salts thereof.
A204. A pharmaceutical composition of phenobarbital or salts thereof that has been reconstituted from a lyophilized pharmaceutical composition of phenobarbital or salts thereof having an alcohol content in the range from about 5000 ppm to about 66000 ppm, wherein the pharmaceutical composition is obtained by a process comprising: dissolving phenobarbital or salts thereof in water to obtain an aqueous solution having a concentration 10-200 mg/ml; lyophilizing the aqueous solution to obtain lyophilized pharmaceutical composition of phenobarbital or salts thereof; and reconstituting the lyophilized pharmaceutical composition to obtain the pharmaceutical composition of phenobarbital or salts thereof.
A205. A lyophilized pharmaceutical composition comprising phenobarbital sodium and ethanol, wherein ethanol is present in an amount sufficient to inhibit degradation of phenobarbital sodium, such that the amount of total impurities present following 36 months of storage at 20-25° C. does not exceed 0.2%;
   wherein the amount of ethanol sufficient to inhibit degradation of phenobarbital sodium is in the range from about 12000 ppm to about 25000 ppm;
   and wherein the pharmaceutical composition is free of benzyl alcohol and propylene glycol.
A206. An aqueous solution for injection comprising phenobarbital sodium and ethanol, wherein ethanol is present in an amount sufficient to inhibit degradation of phenobarbital sodium, such that the amount of total impurities present following 12 hours of storage at 20-25° C. or following 36 hours of storage at 2-8° C. does not exceed 0.2%;
   wherein the amount of ethanol sufficient to inhibit degradation of phenobarbital sodium is in the range from about 12000 ppm to about 25000 ppm;
   wherein phenobarbital sodium is present in a concentration from 10-200 mg/ml;
   wherein the aqueous solution is reconstituted from the lyophilized pharmaceutical composition of phenobarbital sodium;
   and wherein the aqueous solution is free of benzyl alcohol and propylene glycol.
A207. A process for the preparation of the pharmaceutical composition of phenobarbital or salts thereof, the process comprises dissolving phenobarbital or salt thereof in water to obtain an aqueous solution having a concentration 10-200 mg/ml and lyophilizing the aqueous solution to obtain lyophilized pharmaceutical composition, wherein phenobarbital or salts thereof has an alcohol content in the range from about 5000 ppm to about 66000 ppm.
A208. A lyophilized pharmaceutical composition of phenobarbital sodium, wherein the composition has an ethanol content in the range from about 12000 ppm to about 25000 ppm; wherein the composition is stable up to 36 months of storage at 20-25° C. such that the amount of total impurities present following 36 months of storage at 20-25° C. does not exceed 0.2%; and wherein the composition is free of benzyl alcohol and propylene glycol.
A209. An aqueous solution for injection of phenobarbital sodium, wherein the aqueous solution has an ethanol content in the range from about 12000 ppm to about 25000 ppm; wherein the aqueous solution is stable up to 12 hours of storage at 20-25° C. or 36 hours of storage at 2-8° C. such that the amount of total impurities present following 12 hours of storage at 20-25° C. or following 36 hours of storage at 2-8° C. does not exceed 0.2%; wherein the aqueous solution is reconstituted from the lyophilized pharmaceutical composition of phenobarbital sodium; and wherein the aqueous solution is free of benzyl alcohol and propylene glycol.
A210. A method for the preparation of the lyophilized pharmaceutical composition of phenobarbital sodium comprising: (a) dissolving phenobarbital sodium in water to obtain a pre-lyophilization solution having a concentration between 10 mg/ml to 60 mg/ml of phenobarbital sodium; and (b) lyophilizing the pre-lyophilized solution to obtain a lyophilized pharmaceutical composition.
A211. A method for the preparation of the lyophilized pharmaceutical composition of phenobarbital sodium comprising: (a) dissolving phenobarbital sodium in water to obtain a pre-lyophilization solution having a concentration between 10 mg/ml to 60 mg/ml of phenobarbital sodium; and (b) lyophilizing the pre-lyophilized solution to obtain a lyophilized pharmaceutical composition; wherein step (b) comprises: (i) freezing a pre-lyophilized solution of step (a) to a temperature from about 5° C. to about −45° C. to form a frozen solution; (ii) holding the frozen solution at or below −45° C., for about 200 min to about 500 min; (iii) evacuating and ramping the frozen solution to a primary drying temperature to form a dried solution, wherein the primary drying temperature is higher than the collapse temperature of phenobarbital sodium; (iv) holding for less than about 1000 min, under a pressure of about 35 to about 120 mTorr; (v) ramping the dried solution to a secondary drying temperature from about 20° C. to about 45° C.; and (vi) holding for about 200 min to about 400 min, under a pressure of about 35 to about 60 mTorr; to form a lyophilized composition of phenobarbital sodium.
A212. A method for the preparation of the lyophilized pharmaceutical composition of phenobarbital sodium comprising: (a) dissolving phenobarbital sodium in water to obtain a pre-lyophilization solution having a concentration between 10 mg/ml to 60 mg/ml of phenobarbital sodium; and (b) lyophilizing the pre-lyophilized solution to obtain a lyophilized pharmaceutical composition; wherein step (b) comprises: (i) freezing a pre-lyophilization solution of step (a) to a temperature of about −45° C. to form a frozen solution; (ii) holding the frozen solution at or below −45° C., for about 300 min; (iii) evacuating and ramping the frozen solution to a primary drying temperature of about 20° C. to form a dried solution; (iv) holding for less than about 900 min, under a pressure of less than about 60 mTorr; (v) ramping the dried solution to a secondary drying temperature of about 45° C.; and (vi) holding for about 330 min, under a pressure of about 50 mTorr; to form a lyophilized pharmaceutical composition of phenobarbital sodium.
A213. A lyophilized pharmaceutical composition of phenobarbital sodium, prepared by a method according to A210 to A212.
A214. An injectable formulation comprising phenobarbital sodium, reconstituted from the lyophilized pharmaceutical composition A213 in a sterile carrier suitable for intravenous administration.
A215. A lyophilized pharmaceutical composition of phenobarbital sodium having one or more of: a percent porosity in the range from about 20% to about 50%; an ethanol content in the range from about 12000 ppm to about 25000 ppm; total impurities in an amount of less than 0.2% following 36 months of storage at about 20° C. to about 25° C.; free of benzyl alcohol and propylene glycol; or lyophilized phenobarbital sodium in an amount from 10 mg/vial to 100 mg/vial.

What is claimed is:

1. A method for the preparation of the lyophilized pharmaceutical composition of phenobarbital sodium comprising:
(a) dissolving phenobarbital sodium in water to obtain a pre-lyophilization solution having a concentration between 10 mg/ml to 60 mg/ml of phenobarbital sodium; and
(b) lyophilizing the pre-lyophilized solution to obtain a lyophilized pharmaceutical composition.

2. The method according to claim 1, wherein the lyophilized pharmaceutical composition has a percent porosity in the range from about 20% to about 50%.

3. The method according to claim 1, wherein the lyophilized pharmaceutical composition has a percent porosity in the range from about 25% to about 45%.

4. The method according to claim 1, wherein the lyophilized pharmaceutical composition further comprises an ethanol content in the range from about 12000 ppm to about 25000 ppm.

5. The method according to claim 1, wherein the lyophilized pharmaceutical composition comprises total impurities in an amount of less than 0.2% following 36 months of storage at about 20° C. to about 25° C.

6. The method according to claim 1, wherein the lyophilized pharmaceutical composition is free of benzyl alcohol and propylene glycol.

7. The method according to claim 1, wherein step (b) comprises:
(i) freezing a pre-lyophilized solution of step (a) to a temperature from about 5° C. to about −45° C. to form a frozen solution;
(ii) holding the frozen solution at or below −45° C., for about 200 min to about 500 min;
(iii) evacuating and ramping the frozen solution to a primary drying temperature to form a dried solution, wherein the primary drying temperature is higher than the collapse temperature of phenobarbital sodium;
(iv) holding for less than about 1000 min, under a pressure of about 35 to about 120 mTorr;
(v) ramping the dried solution to a secondary drying temperature from about 20° C. to about 45° C.; and
(vi) holding for about 200 min to about 400 min, under a pressure of about 35 to about 60 mTorr; to form a lyophilized composition of phenobarbital sodium.

8. The method according to claim 7, wherein the primary drying temperature is about 20° C.

9. The method according to claim 1, wherein step (b) comprises:
(i) freezing a pre-lyophilization solution of step (a) to a temperature of about −45° C. to form a frozen solution;
(ii) holding the frozen solution at or below −45° C., for about 300 min;
(iii) evacuating and ramping the frozen solution to a primary drying temperature of about 20° C. to form a dried solution;
(iv) holding for less than about 900 min, under a pressure of less than about 60 mTorr;
(v) ramping the dried solution to a secondary drying temperature of about 45° C.; and
(vi) holding for about 330 min, under a pressure of about 50 mTorr; to form a lyophilized pharmaceutical composition of phenobarbital sodium.

10. A lyophilized pharmaceutical composition of phenobarbital sodium, prepared by a method according to claim 1.

11. An injectable formulation comprising phenobarbital sodium, reconstituted from the lyophilized pharmaceutical composition according to claim 10 in a sterile carrier suitable for intravenous administration.

12. A lyophilized pharmaceutical composition of phenobarbital sodium having a percent porosity in the range from about 20% to about 50%.

13. The lyophilized pharmaceutical composition according to claim 12, further comprising an ethanol content in the range from about 12000 ppm to about 25000 ppm.

14. The lyophilized pharmaceutical composition according to claim 12, comprising total impurities in an amount of less than 0.2% following 36 months of storage at about 20° C. to about 25° C.

15. The lyophilized pharmaceutical composition according to claim 12, which is free of benzyl alcohol and propylene glycol.

16. The lyophilized pharmaceutical composition according to claim 12, comprising lyophilized phenobarbital sodium in an amount from 10 mg/vial to 100 mg/vial.

17. The lyophilized pharmaceutical composition according to claim 16, comprising lyophilized phenobarbital sodium in an amount of 10 mg/vial.

18. The lyophilized pharmaceutical composition according to claim 16, comprising lyophilized phenobarbital sodium in an amount of 100 mg/vial.

19. A lyophilized pharmaceutical composition of phenobarbital sodium having one or more of:
(a) a percent porosity in the range from about 20% to about 50%;
(b) an ethanol content in the range from about 12000 ppm to about 25000 ppm;
(c) total impurities in an amount of less than 0.2% following 36 months of storage at about 20° C. to about 25° C.;
(d) free of benzyl alcohol and propylene glycol; or
(e) lyophilized phenobarbital sodium in an amount from 10 mg/vial to 100 mg/vial.

20. The lyophilized pharmaceutical composition according to claim 12, wherein the impurities are selected from the group consisting of 2-phenyl-2-ethyl acetyl urea, 2-phenyl-2-ethyl-malonamide, α-phenylbutyrylguanidine, 2-phenylbutyric acid and 5-methyl-5-phenylbarbituric acid.

21. The lyophilized pharmaceutical composition according to claim 12, wherein the lyophilized pharmaceutical composition is reconstituted with water for injection, an aqueous saline or an aqueous dextrose solution.

22. The lyophilized pharmaceutical composition according to claim 12, wherein the phenobarbital sodium is present in a concentration of 10-200 mg/ml.

23. The lyophilized pharmaceutical composition according to claim 12, wherein the pharmaceutical composition has an osmolality below 500 mOsm/kg.

24. The lyophilized pharmaceutical composition according to claim 12 prepared by a process comprising: (a) dissolving phenobarbital sodium in water to obtain a pre-lyophilization solution having a concentration between 10 mg/ml to 60 mg/ml of phenobarbital sodium; and (b) lyophilizing the pre-lyophilized solution of step (a).

25. A pharmaceutical composition of phenobarbital sodium made by a method comprising reconstituting a lyophilized pharmaceutical composition of according to claim 12.

26. A method for the treatment of neonatal seizure in newborn infants of 2 weeks of age or younger in need thereof, comprising administering the pharmaceutical composition according to claim 25.

27. The method according to claim 26, wherein the method comprises reconstituting the lyophilized pharmaceutical composition immediately prior to the administration.

28. The method according to claim 27, wherein the lyophilized pharmaceutical composition is reconstituted with water for injection, an aqueous saline or an aqueous dextrose solution.
